SEQUENCE LISTING (70 sequences)

SEQ ID NO: 1 - DNA, 3927 bp, Homo sapiens
  CDS (126)...(3665): Sig-Peptide (126-179)
gaattccggcccccgtgtctgggcgtccgcctcctggcctcctggctgaggggaagctgagtgggccacggcccatgtgtcgcactcgcctcggctcccacacagccgcctctgctccagcaaggatgtggctcttccacactctgctctgcatagccagcctggccctgctggccgctttcaatgtggatgtggcccggccctggctcacgcccaagggaggtgcccctttcgtgctcagctcccttctgcaccaagaccccagcaccaaccagacctggctcctggtcaccagccccagaaccaagaggacaccagggcccctccatcgatgttcccttgtccaggatgaaatcctttgccatcctgtagagcatgtccccatccccaaggggaggcaccggggagtgaccgttgtccggagccaccacggtgttttgatatgcattcaagtgctggtccggcggcctcacagcctcagctcagaactcacaggcacctgtagcctcctgggccctgacctccgtccccaggctcaggccaacttcttcgaccttgaaaatctcctggatccagatgcacgtgtggacactggagactgctacagcaacaaagaaggcggtggagaagacgatgtgaacacagccaggcagcgccgggctctggagaaggaggaggaggaagacaaggaggaggaggaagacgaggaggaggaggaagctggcaccgagattgccatcatcctggatggctcaggaagcattgatcccccagactttcagagagccaaagacttcatctccaacatgatgaggaacttctatgaaaagtgttttgagtgcaactttgccttggtgcagtatggaggagtgatccagactgagtttgaccttcgggacagccaggatgtgatggcctccctcgccagagtccagaacatcactcaagtggggagtgtcaccaagactgcctcagccatgcaacacgtcttagacagcatcttcacctcaagccacggctccaggagaaaggcatccaaggtcatggtggtgctcaccgatggtggcatattcgaggaccccctcaaccttacgacagtcatcaactcccccaaaatgcagggtgttgagcgctttgccattggggtgggagaagaatttaagagtgctaggactgcgagggaactgaacctgatcgcctcagacccggatgagacccatgctttcaaggtgaccaactacatggcgctggatgggctgctgagcaaactgcggtacaacatcatcagcatggaaggcacggttggagacgcccttcactaccagctggcacagattggcttcagtgctcagatcctggatgagcggcaggtgctgctcggcgccgtcggggcctttgactggtccggaggggcgttgctctacgacacacgcagccgccggggccgcttcctgaaccagacagcggcggcggcggcagacgcggaggctgcgcagtacagctacctgggttacgctgtggccgtgctgcacaagacctgcagcctctcctacgtcgcgggggctccacagtacaaacatcatggggccgtgtttgagctccagaaggagggcagagaggccagcttcctgccagtgctggagggagagcagatggggtcctattttggctctgagctgtgccctgtggacattgacatggatggaagcacggacttcttgctggtggctgctccattttaccacgttcatggagaagaaggcagagtctacgtgtaccgtctcagcgagcaggatggttctttctccttggcacgcatactgagtgggcaccccgggttcaccaatgcccgctttggctttgccatggcggctatgggggatctcagtcaggataagctcacagatgtggccatcggggcccccctggaaggttttggggcagatgatggtgccagcttcggcagtgtgtatatctacaatggacactgggacggcctctccgccagcccctcgcagcggatcagagcctccacggtggccccaggactccagtacttcggcatgtccatggctggtggctttgatattagtggcgacggccttgccgacatcaccgtgggcactctgggccaggcggttgtgttccgctcccggcctgtggttcgcctgaaggtctccatggccttcacccccagcgcactgcccatcggcttcaacggcgtcgtgaatgtccgtttatgttttgaaatcagctctgtaaccacagcctctgagtcaggcctccgtgaggcacttctcaacttcacgctggatgtggatgtggggaagcagaggagacggctgcagtgttcagacgtaagaagctgtctgggctgcctgagggagtggagcagcggatcccagctttgtgaggacctcctgctcatgcccacagagggagagctctgtgaggaggactgcttctccaatgccagtgtcaaagtcagctaccagctccagacccctgagggacagacggaccatccccagcccatcctggaccgctacactgagccctttgccatcttccagctgccctatgagaaggcctgcaagaataagctgttttgtgtcgcagaattacagttggccaccaccgtctctcagcaggagttggtggtgggtctcacaaaggagctgaccctgaacattaacctaactaactccggggaagattcctacatgacaagcatggccttgaattaccccagaaacctgcagttgaagaggatgcaaaagcctccctctccaaacattcagtgtgatgaccctcagccggttgcttctgtcctgatcatgaactgcaggattggtcaccccgtcctcaagaggtcatctgctcatgtttcagtcgtttggcagctagaggagaatgcctttccaaacaggacagcagacatcactgtgactgtcaccaattccaatgaaagacggtctttggccaacgagacccacacccttcaattcaggcatggcttcgttgcagttctgtccaaaccatccataatgtacgtgaacacaggccaggggctttctcaccacaaagaattcctcttccatgtacatggggagaacctctttggagcagaataccagttgcaaatttgcgtcccaaccaaattacgaggtctccaggttgcagcagtgaagaagctgacgaggactcaggcctccacggtgtgcacctggagtcaggagcgcgcttgtgcgtacagttcggttcagcatgtggaagaatggcattcagtgagctgtgtcatcgcttcagataaagaaaatgtcaccgtggctgcagagatctcctgggatcactctgaggagttactaaaagatgtaactgaactgcagatccttggtgaaatatctttcaacaaatctctatatgagggactgaatgcagagaaccacagaactaagatcactgtcgtcttcctgaaagatgagaagtaccattctttgcctatcatcattaaaggcagcgttggtggacttctggtgttgatcgtgattctggtcatcctgttcaagtgtggcttttttaaaagaaaatatcaacaactgaacttggagagcatcaggaaggcccagctgaaatcagagaatctgctcgaagaagagaattaggacctgctatccactgggagaggctatcagccagtcctgggacttggagacccagcatcctttgcattactttttccttcaggatgatctagagcagcatggagctgttggtagaatattagtttttaaccatacattgtcccaaaagtgtctgtgcattgtgcaaaaagtaaacttaggaaacatttggtattaaataaatttacacttttctttgcaaaaaaaaaaaaaaaaaaaaaaaaaaaaaaaaaaaaaaaaaaaa

SEQ ID NO: 2 - protein, 1179 aa, Homo sapiens
  SIGNAL (1)...(18)
MWLFHTLLCIASLALLAAFNVDVARPWLTPKGGAPFVLSSLLHQDPSTNQTWLLVTSPRTKRTPGPLHRCSLVQDEILCHPVEHVPIPKGRHRGVTVVRSHHGVLICIQVLVRRPHSLSSELTGTCSLLGPDLRPQAQANFFDLENLLDPDARVDTGDCYSNKEGGGEDDVNTARQRRALEKEEEEDKEEEEDEEEEEAGTEIAIILDGSGSIDPPDFQRAKDFISNMMRNFYEKCFECNFALVQYGGVIQTEFDLRDSQDVMASLARVQNITQVGSVTKTASAMQHVLDSIFTSSHGSRRKASKVMVVLTDGGIFEDPLNLTTVINSPKMQGVERFAIGVGEEFKSARTARELNLIASDPDETHAFKVTNYMALDGLLSKLRYNIISMEGTVGDALHYQLAQIGFSAQILDERQVLLGAVGAFDWSGGALLYDTRSRRGRFLNQTAAAAADAEAAQYSYLGYAVAVLHKTCSLSYVAGAPQYKHHGAVFELQKEGREASFLPVLEGEQMGSYFGSELCPVDIDMDGSTDFLLVAAPFYHVHGEEGRVYVYRLSEQDGSFSLARILSGHPGFTNARFGFAMAAMGDLSQDKLTDVAIGAPLEGFGADDGASFGSVYIYNGHWDGLSASPSQRIRASTVAPGLQYFGMSMAGGFDISGDGLADITVGTLGQAVVFRSRPVVRLKVSMAFTPSALPIGFNGVVNVRLCFEISSVTTASESGLREALLNFTLDVDVGKQRRRLQCSDVRSCLGCLREWSSGSQLCEDLLLMPTEGELCEEDCFSNASVKVSYQLQTPEGQTDHPQPILDRYTEPFAIFQLPYEKACKNKLFCVAELQLATTVSQQELVVGLTKELTLNINLTNSGEDSYMTSMALNYPRNLQLKRMQKPPSPNIQCDDPQPVASVLIMNCRIGHPVLKRSSAHVSVVWQLEENAFPNRTADITVTVTNSNERRSLANETHTLQFRHGFVAVLSKPSIMYVNTGQGLSHHKEFLFHVHGENLFGAEYQLQICVPTKLRGLQVAAVKKLTRTQASTVCTWSQERACAYSSVQHVEEWHSVSCVIASDKENVTVAAEISWDHSEELLKDVTELQILGEISFNKSLYEGLNAENHRTKITVVFLKDEKYHSLPIIIKGSVGGLLVLIVILVILFKCGFFKRKYQQLNLESIRKAQLKSENLLEEEN

SEQ ID NO: 3 - DNA, 369 bp, Homo sapiens
gaggtgcagctggtgcagtctggagcagaggtgaaaaagcccggggagtctctgaagatctcctgtaagggttctggatacagctttaccagctattggatcggctgggtgcgccagatgcccgggaaaggcctggagtggatggggatcatctatcctggtgactctggtcccagatacagcccgtccttccaaggccaggtcaccatctcagccgacaagtccatcagcaccgcctacctgcagtggagcagcctgaaggcctcggacaccgccatgtattactgtgcgcgactgtcgtataccagcacctggtacccgtactactttgactactggggccagggaaccctggtcaccgtctcctca

SEQ ID NO: 4 - protein, 123 aa, Homo sapiens
  SITE (31)...(35): CDR1
EVQLVQSGAEVKKPGESLKISCKGSGYSFTSYWIGWVRQMPGKGLEWMGIIYPGDSGPRYSPSFQGQVTISADKSISTAYLQWSSLKASDTAMYYCARLSYTSTWYPYYFDYWGQGTLVTVSS

SEQ ID NO: 5 - protein, 5 aa, Homo sapiens
SYWIG

SEQ ID NO: 6 - protein, 17 aa, Homo sapiens
IIYPGDSGPRYSPSFQG

SEQ ID NO: 7 - protein, 14 aa, Homo sapiens
LSYTSTWYPYYFDY

SEQ ID NO: 8 - DNA, 324 bp, Homo sapiens
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccggggacgttcggccaagggaccaaggtggaaatcaaa

SEQ ID NO: 9 - protein, 108 aa, Homo sapiens
  SITE (24)...(34): CDR1
EIVLTQSPATLSLSPGERATLSCRASQSVSSYLAWYQQKPGQAPRLLIYDASNRATGIPARFSGSGSGTDFTLTISSLEPEDFAVYYCQQRSNWPPGTFGQGTKVEIK

SEQ ID NO: 10 - protein, 11 aa, Homo sapiens
RASQSVSSYLA

SEQ ID NO: 11 - protein, 7 aa, Homo sapiens
DASNRAT

SEQ ID NO: 12 - protein, 10 aa, Homo sapiens
QQRSNWPPGT

SEQ ID NO: 13 - DNA, 354 bp, Homo sapiens
gaggtgcagttggtggagtctgggggaggcttggtccagcctggagggtccctgagactctcctgtgcagcctctggattcacctttagtaacttttggatgagctgggtccgccaggctccagggaaagggctggagtggatggccaacataaagcaagatggaagtgagaaatactatgtggactctgtgaagggccgattcaccatctccagagacaacgccaagagctcactgtttctgcaaatgaacagcctgagagtcgacgacacggctgtatatttctgtgcgggggattactatgattcggggagtttctactggggccagggaaccctggtcaccgtctcctca

SEQ ID NO: 14 - protein, 118 aa, Homo sapiens
  SITE (31)...(35): CDR1
EVQLVESGGGLVQPGGSLRLSCAASGFTFSNFWMSWVRQAPGKGLEWMANIKQDGSEKYYVDSVKGRFTISRDNAKSSLFLQMNSLRVDDTAVYFCAGDYYDSGSFYWGQGTLVTVSS

SEQ ID NO: 15 - protein, 5 aa, Homo sapiens
NFWMS

SEQ ID NO: 16 - protein, 17 aa, Homo sapiens
NIKQDGSEKYYVDSVKG

SEQ ID NO: 17 - protein, 9 aa, Homo sapiens
DYYDSGSFY

SEQ ID NO: 18 - DNA, 324 bp, Homo sapiens
gaaattgtgttgacacagtctccagccaccctgtctttgtctccaggggaaagagccaccctctcctgcagggccagtcagagtgttaggagcaacttagcctggtaccaacagaaacctggccaggctcccaggctcctcatctatgatgcatccaacagggccattggcatcccagccaggttcagtggcagtgggtctgggacagacttcactctcaccatcagcagtttagagcctgaagattttgtagtttattactgtcagcagcgtagcaactggcctccgtggacgttcggccaagggaccaaggtggaaatcaaa

SEQ ID NO: 19 - protein, 108 aa, Homo sapiens
  SITE (24)...(34): CDR1
EIVLTQSPATLSLSPGERATLSCRASQSVRSNLAWYQQKPGQAPRLLIYDASNRAIGIPARFSGSGSGTDFTLTISSLEPEDFVVYYCQQRSNWPPWTFGQGTKVEIK

SEQ ID NO: 20 - protein, 11 aa, Homo sapiens
RASQSVRSNLA

SEQ ID NO: 21 - protein, 7 aa, Homo sapiens
DASNRAI

SEQ ID NO: 22 - protein, 10 aa, Homo sapiens
QQRSNWPPWT

SEQ ID NO: 23 - DNA, 384 bp, Homo sapiens
caggtgcagctgcaggagtcgggcccaggactggtgaagccttcggagaccctgtccctcacctgcactgtctctggtggctccgtcagtagttactattggagctggatccggcagcccccagggaagggactggagtggattggccatatctattacagtgggaataccaactacaacccctccctcaagagtcgagtcaccatatcagtagacacgtccaagaatcagttctccctgaaactgagctctgtgaccgctgcggacacggccgtgtatttttgtgcgagagatagatggaattattatgatagtagtcccggctattattattactacggtatggacgtctggggccaagggaccacggtcaccgtcagctca

SEQ ID NO: 24 - protein, 128 aa, Homo sapiens
  SITE (31)...(35): CDR1
QVQLQESGPGLVKPSETLSLTCTVSGGSVSSYYWSWIRQPPGKGLEWIGHIYYSGNTNYNPSLKSRVTISVDTSKNQFSLKLSSVTAADTAVYFCARDRWNYYDSSPGYYYYYGMDVWGQGTTVTVSS

SEQ ID NO: 25 - protein, 5 aa, Homo sapiens
SYYWS

SEQ ID NO: 26 - protein, 16 aa, Homo sapiens
HIYYSGNTNYNPSLKS

SEQ ID NO: 27 - protein, 20 aa, Homo sapiens
DRWNYYDSSPGYYYYYGMDV

SEQ ID NO: 28 - DNA, 321 bp, Homo sapiens
gacatccagatgacccagtctccatcctccctgtctgcatctgtaggagacagagtcaccatcacttgccgggcgagtcagggcattagaaatgatttaggctggtatcagcaaaaaccagggaaagcccctaagcgcctaatctttgctgcatcccatttgcaaagtggagtcccttcaaggttcagcggcagtggatctgggacagagttcactctcacaatcagcagcctgcagcctgaagattttgcaacttattactgtcaacagcataatagttccccattcactttcggccctgggaccagagtggatatcaaa

SEQ ID NO: 29 - protein, 107 aa, Homo sapiens
  SITE (24)...(34): CDR1
DIQMTQSPSSLSASVGDRVTITCRASQGIRNDLGWYQQKPGKAPKRLIFAASHLQSGVPSRFSGSGSGTEFTLTISSLQPEDFATYYCQQHNSSPFTFGPGTRVDIK

SEQ ID NO: 30 - protein, 11 aa, Homo sapiens
RASQGIRNDLG

SEQ ID NO: 31 - protein, 7 aa, Homo sapiens
AASHLQS

SEQ ID NO: 32 - protein, 9 aa, Homo sapiens
QQHNSSPFT

SEQ ID NO: 33 - DNA, 2808 bp, Homo sapiens
  CDS (109)...(2745)
ggaaagcacctgtgagcttggcaagtcagttcagagctccagcccgctccagcccggcccgacccgaccgcacccggcgcctgcctcgctcgggctccccggccagccatgggcccttggagccgcagcctctcgggcctgctgctgctgctgaggtctcctcttggctctcaggagcggagccctcctccctgtttgacgcgagagctacacgttcacggtgccccggcgccacctgagaagaggccgcgtctgggcagagtgaattttgaagattgcaccggtcgacaaaggacagctattttcctgacaccgattccgaaagtgggcacagatggtgtgattacagtcaaaaggcctctacggtttcataacccaacagatccatttcttggtctacgctgggactccacctacagaaagttttccaccaaagtcacgctgaatacagtggggcaccaccaccgccccccgccccatcaggcctccgtttctggaatccaagcagaattgctcacatttcccaactcctctcctggcctcagaagacagaagagagactgggttattcctcccatcagctgcccagaaaatgaaaaaggcccatttcctaaaaacctggttcagatcaaatccaacaaagacaaagaaggcaaggttttctacagcatcactggccaaggagctgacacaccccctgttggtgtctttattattgaaagagaaacaggatggctgaaggtgacagagcctctggatagagaacgcattgccacatacactctcttctctcacgctgtgtcatccaacgggaatgcagttgaggatccaatggagattttgatcacggtaaccgatcagaatgacaacaagcccgaattcacccaggaggtctttaaggggtctgtcatggaaggtgctcttccaggaacctctgtgatggaggtcacagccacagacgcggacgatgatgtgaacacctacaatgccgccatcgcttacaccatcctcagccaagatcctgagctccctgacaaaaatatgttcaccattaacaggaacacaggagtcatcagtgtggtcaccactgggctggaccgagagagtttccctacgtataccctggtggttcaagctgctgaccttcaaggtgaggggttaagcacaacagcaacagctgtgatcacagtcactgacaccaacgataatcctccgatcttcaatcccaccacgtacaagggtcaggtgcctgagaacgaggctaacgtcgtaatcaccacactgaaagtgactgatgctgatgcccccaataccccagcgtgggaggctgtatacaccatattgaatgatgatggtggacaatttgtcgtcaccacaaatccagtgaacaacgatggcattttgaaaacagcaaagggcttggattttgaggccaagcagcagtacattctacacgtagcagtgacgaatgtggtaccttttgaggtctctctcaccacctccacagccaccgtcaccgtggatgtgctggatgtgaatgaaggccccatctttgtgcctcctgaaaagagagtggaagtgtccgaggactttggcgtgggccaggaaatcacatcctacactgcccaggagccagacacatttatggaacagaaaataacatatcggatttggagagacactcgcaactggctggagattaatccggacactggtgccatttccactcgggctgagctggacagggaggattttgagcacgtgaagaacagcacgtacacagccctaatcatagctacagacaatggttctccagttgctactggaacagggacacttctgctgatcctgtctgatgtgaatgacaacgcccccataccagaacctcgaactatattcttctgtgagaggaatccaaagcctcaggtcataaacattcatgatgcagaccttcctcccaatacatctcccttcacagcagaactaacacacgggcgagtgcccaactggaccattcagtacaacgacccaacccaagaatctatcattttgaagccaaagatggccttagaggtgggtgactacaaaatcaatctcaagctcatggataaccagaataaagaccaagtgaccaccttagaggtcagcgtgtgtgactgtgaaggggccgccggcgtctgtaggaaggcacagcctgtcgaagcaggattgcaaattcctgccattctggggattcttggaggaattcttgctttgctaattctgattctgctgctcttgctgtttcttcggaggagagcggtggtcaaagagcccttactgcccccagaggatgacacccgggacaacgtttattactatgatgaagaaggaggcggagaagaggaccaggactttgacttgagccagctgcacaggggcctggacgctcggcctgaagtgactcgtaacgacgttgcaccaaccctcatgagtgtcccccggtatcttccccgccctgccaatcccgatgaaattggaaattttattgatgaaaatctgaaagcggctgatactgaccccacagccccgccttatgattctctgctcgtgtttgactatgaaggaagcggttccgaagctgctagtctgagctccctgaactcctcagagtcagacaaagaccaggactatgactacttgaacgaatggggcaatccgttcaagaagctggctgacatgtacggaggcggcgaggaccactaggggactcgagagaggcggcccagaccatgtgcagaaatgcagaaatcagcgttctggtgtttt

SEQ ID NO: 34 - protein, 878 aa, Homo sapiens [sequence omitted]

SEQ ID NO: 35 - DNA, 3567 bp, Homo sapiens
  CDS (411)...(3527)
cgccatcccgcgctctgcggactgggaggcccgggccaggacgcgagtctgcgcagccgaggttccccagcgccccctgcagccgcgcgtaggcagagacggagcccggccctgcgcctccgcaccacgcccgggaccccacccagcggcccgtacccggagaagcagcgcgagcacccgaagctcccggctcggcggcagaaaccgggagtggggccgggcgagtgcgcggcatcccaggccggcccgaacgtccgcccgcggtgggccgacttcccctcctcttccctctctccttcctttagcccgctggcgccggacacgctgcgcctcatctcttggggcgttcttccccgttggccaaccgtcgcatcccgtgcaactttggggtagtggccgcttagtgttgaatgttccccaccgagagcgcatggcttgggaagcgaggcgcgaacccgggccccgaagccgccgtccgggagacggtgatgctgttgctgtgcctgggggtcccgaccggccgcccctacaacgtggacactgagagcgcgctgctttaccagggcccccacaacacgctgttcggctactcggtcgtgctgcacagccacggggcgaaccgatggctcctagtgggtgcgcccactgccaactggctcgccaacgcttcagtgatcaatcccggggcgatttacagatgcaggatcggaaagaatcccggccagacgtgcgaacagctccagctgggtagccctaatggagaaccttgtggaaagacttgtttggaagagagagacaatcagtggttgggggtcacactttccagacagccaggagaaaatggatccatcgtgacttgtgggcatagatggaaaaatatattttacataaagaatgaaaataagctccccactggtggttgctatggagtgccccctgatttacgaacagaactgagtaaaagaatagctccgtgttatcaagattatgtgaaaaaatttggagaaaattttgcatcatgtcaagctggaatatccagtttttacacaaaggatttaattgtgatgggggccccaggatcatcttactggactggctctctttttgtctacaatataactacaaataaatacaaggcttttttagacaaacaaaatcaagtaaaatttggaagttatttaggatattcagtcggagctggtcattttcggagccagcatactaccgaagtagtcggaggagctcctcaacatgagcagattggtaaggcatatatattcagcattgatgaaaaagaactaaatatcttacatgaaatgaaaggtaaaaagcttggatcgtactttggagcttctgtctgtgctgtggacctcaatgcagatggcttctcagatctgctcgtgggagcacccatgcagagcaccatcagagaggaaggaagagtgtttgtgtacatcaactctggctcgggagcagtaatgaatgcaatggaaacaaacctcgttggaagtgacaaatatgctgcaagatttggggaatctatagttaatcttggcgacattgacaatgatggctttgaagatgttgctatcggagctccacaagaagatgacttgcaaggtgctatttatatttacaatggccgtgcagatgggatctcgtcaaccttctcacagagaattgaaggacttcagatcagcaaatcgttaagtatgtttggacagtctatatcaggacaaattgatgcagataataatggctatgtagatgtagcagttggtgcttttcggtctgattctgctgtcttgctaaggacaagacctgtagtaattgttgacgcttctttaagccaccctgagtcagtaaatagaacgaaatttgactgtgttgaaaatggatggccttctgtgtgcatagatctaacactttgtttctcatataagggcaaggaagttccaggttacattgttttgttttataacatgagtttggatgtgaacagaaaggcagagtctccaccaagattctatttctcttctaatggaacttctgacgtgattacaggaagcatacaggtgtccagcagagaagctaactgtagaacacatcaagcatttatgcggaaagatgtgcgggacatcctcaccccaattcagattgaagctgcttaccaccttggtcctcatgtcatcagtaaacgaagtacagaggaattcccaccacttcagccaattcttcagcagaagaaagaaaaagacataatgaaaaaaacaataaactttgcaaggttttgtgcccatgaaaattgttctgctgatttacaggtttctgcaaagattgggtttttgaagccccatgaaaataaaacatatcttgctgttgggagtatgaagacattgatgttgaatgtgtccttgtttaatgctggagatgatgcatatgaaacgactctacatgtcaaactacccgtgggtctttatttcattaagattttagagctggaagagaagcaaataaactgtgaagtcacagataactctggcgtggtacaacttgactgcagtattggctatatatatgtagatcatctctcaaggatagatattagctttctcctggatgtgagctcactcagcagagcggaagaggacctcagtatcacagtgcatgctacctgtgaaaatgaagaggaaatggacaatctaaagcacagcagagtgactgtagcaatacctttaaaatatgaggttaagctgactgttcatgggtttgtaaacccaacttcatttgtgtatggatcaaatgatgaaaatgagcctgaaacgtgcatggtggagaaaatgaacttaactttccatgttatcaacactggcaatagtatggctcccaatgttagtgtggaaataatggtaccaaattcttttagcccccaaactgataagctgttcaacattttggatgtccagactactactggagaatgccactttgaaaattatcaaagagtgtgtgcattagagcagcaaaagagtgcaatgcagaccttgaaaggcatagtccagttcttgtccaagactgataagaggctattgtactgcataaaagctgatccacattgtttaaatttcttgtgtaattttgggaaaatggaaagtggaaaagaagccagtgttcatatccaactggaaggccggccatccattttagaaatggatgagacttcagcactcaagtttgaaataagagcaacaggttttccagagccaaatccaagagtaattgaactaaacaaggatgagaatgttgcgcatgttctactggaaggactacatcatcaaagacccaaacgttatttcaccatagtgattatttcaagtagcttgctacttggacttattgtacttctgttgatctcatatgttatgtggaaggctggcttctttaaaagacaatacaaatctatcctacaagaagaaaacagaagagacagttggagttatatcaacagtaaaagcaatgatgattaaggacttctttcaaattgagagaatggaaaacagcccgccc

SEQ ID NO: 36 - protein, 1038 aa, Homo sapiens [sequence omitted]

SEQ ID NO: 37 - DNA, 2742 bp, Homo sapiens
  CDS (114)...(2510)
agcccagagagaaagtctgacttgccccacagccagtgagtgactgcagcagcaccagaatctggtctgtttcctgtttggctcttctaccactacggcttgggatctcgggcatggtggctttgccaatggtccttgttttgctgctggtcctgagcagaggtgagagtgaattggacgccaagatcccatccacaggggatgccacagaatggcggaatcctcacctgtccatgctggggtcctgccagccagccccctcctgccagaagtgcatcctctcacaccccagctgtgcatggtgcaagcaactgaacttcaccgcgtcgggagaggcggaggcgcggcgctgcgcccgacgagaggagctgctggctcgaggctgcccgctggaggagctggaggagccccgcggccagcaggaggtgctgcaggaccagccgctcagccagggcgcccgcggagagggtgccacccagctggcgccgcagcgggtccgggtcacgctgcggcctggggagccccagcagctccaggtccgcttccttcgtgctgagggatacccggtggacctgtactaccttatggacctgagctactccatgaaggacgacctggaacgcgtgcgccagctcgggcacgctctgctggtccggctgcaggaagtcacccattctgtgcgcattggttttggttcctttgtggacaaaacggtgctgccctttgtgagcacagtaccctccaaactgcgccacccctgccccacccggctggagcgctgccagtcaccattcagctttcaccatgtgctgtccctgacgggggacgcacaagccttcgagcgggaggtggggcgccagagtgtgtccggcaatctggactcgcctgaaggtggcttcgatgccattctgcaggctgcactctgccaggagcagattggctggagaaatgtgtcccggctgctggtgttcacttcagacgacacattccatacagctggggacgggaagttgggcggcattttcatgcccagtgatgggcactgccacttggacagcaatggcctctacagtcgcagcacagagtttgactacccttctgtgggtcaggtagcccaggccctctctgcagcaaatatccagcccatctttgctgtcaccagtgccgcactgcctgtctaccaggagctgagtaaactgattcctaagtctgcagttggggagctgagtgaggactccagcaacgtggtacagctcatcatggatgcttataatagcctgtcttccactgtgacccttgaacactcttcactccctcctggggtccacatttcttacgaatcccagtgtgagggtcctgagaagagggagggtaaggctgaggatcgaggacagtgcaaccacgtccgaatcaaccagacggtgactttctgggtttctctccaagccacccactgcctcccagagccccatctcctgaggctccgggcccttggcttctcagaggagctgattgtggagttgcacacgctgtgtgactgtaattgcagtgacacccagccccaggctccccactgcagtgatggccagggacacctacaatgtggtgtatgcagctgtgcccctggccgcctaggtcggctctgtgagtgctctgtggcagagctgtcctccccagacctggaatctgggtgccgggctcccaatggcacagggcccctgtgcagtggaaagggtcactgtcaatgtggacgctgcagctgcagtggacagagctctgggcatctgtgcgagtgtgacgatgccagctgtgagcgacatgagggcatcctctgcggaggctttggtcgctgccaatgtggagtatgtcactgtcatgccaaccgcacgggcagagcatgcgaatgcagtggggacatggacagttgcatcagtcccgagggagggctctgcagtgggcatggacgctgcaaatgcaaccgctgccagtgcttggacggctactatggtgctctatgcgaccaatgcccaggctgcaagacaccatgcgagagacaccgggactgtgcagagtgtggggccttcaggactggcccactggccaccaactgcagtacagcttgtgcccataccaatgtgaccctggccttggcccctatcttggatgatggctggtgcaaagagcggaccctggacaaccagctgttcttcttcttggtggaggatgacgccagaggcacggtcgtgctcagagtgagaccccaagaaaagggagcagaccacacgcaggccattgtgctgggctgcgtagggggcatcgtggcagtggggctggggctggtcctggcttaccggctctcggtggaaatctatgaccgccgggaatacagtcgctttgagaaggagcagcaacaactcaactggaagcaggacagtaatcctctctacaaaagtgccatcacgaccaccatcaatcctcgctttcaagaggcagacagtcccactctctgaaggagggagggacacttacccaaggctcttctccttggaggacagtgggaactggagggtgagaggaagggtgggtctgtaagaccttggtaggggactaattcactggcgaggtgcggccaccaccctacttcattttcagagtgacacccaagagggctgcttcccatgcctgcaaccttgcatccatctgggctaccccacccaagtatacaataaagtcttacctcag

SEQ ID NO: 38 - protein, 798 aa, Homo sapiens
MVALPMVLVLLLVLSRGESELDAKIPSTGDATEWRNPHLSMLGSCQPAPSCQKCILSHPSCAWCKQLNFTASGEAEARRCARREELLARGCPLEELEEPRGQQEVLQDQPLSQGARGEGATQLAPQRVRVTLRPGEPQQLQVRFLRAEGYPVDLYYLMDLSYSMKDDLERVRQLGHALLVRLQEVTHSVRIGFGSFVDKTVLPFVSTVPSKLRHPCPTRLERCQSPFSFHHVLSLTGDAQAFEREVGRQSVSGNLDSPEGGFDAILQAALCQEQIGWRNVSRLLVFTSDDTFHTAGDGKLGGIFMPSDGHCHLDSNGLYSRSTEFDYPSVGQVAQALSAANIQPIFAVTSAALPVYQELSKLIPKSAVGELSEDSSNVVQLIMDAYNSLSSTVTLEHSSLPPGVHISYESQCEGPEKREGKAEDRGQCNHVRINQTVTFWVSLQATHCLPEPHLLRLRALGFSEELIVELHTLCDCNCSDTQPQAPHCSDGQGHLQCGVCSCAPGRLGRLCECSVAELSSPDLESGCRAPNGTGPLCSGKGHCQCGRCSCSGQSSGHLCECDDASCERHEGILCGGFGRCQCGVCHCHANRTGRACECSGDMDSCISPEGGLCSGHGRCKCNRCQCLDGYYGALCDQCPGCKTPCERHRDCAECGAFRTGPLATNCSTACAHTNVTLALAPILDDGWCKERTLDNQLFFFLVEDDARGTVVLRVRPQEKGADHTQAIVLGCVGGIVAVGLGLVLAYRLSVEIYDRREYSRFEKEQQQLNWKQDSNPLYKSAITTTINPRFQEADSPTL

SEQ ID NO: 39 - protein, 30 aa, Unknown
  acidic peptide
AQLEKELQALEKENAQLEWELQALEKELAQ

SEQ ID NO: 40 - protein, 30 aa, Unknown
  basic peptide
AQLKKKLQALKKKNAQLKWKLQALKKKLAQ

SEQ ID NO: 41 - protein, 6 aa, Artificial Sequence
  amino acid linker sequence
GGSTGG

SEQ ID NO: 42 - DNA, 31 bp, Artificial Sequence
  Synthetic oligonucleotide
gcactagtccaccatgggcccttggagccgc

SEQ ID NO: 43 - DNA, 26 bp, Artificial Sequence
  Synthetic oligonucleotide
ccctcgagaggctgtgccttcctaca

SEQ ID NO: 44 - DNA, 23 bp, Artificial Sequence
  Synthetic oligonucleotide
atctcgagcccaaatcttgtgac

SEQ ID NO: 45 - DNA, 28 bp, Artificial Sequence
  Synthetic oligonucleotide
tagcggccgctcatttacccggagacag

SEQ ID NO: 46 - DNA, 21 bp, Artificial Sequence
  Synthetic oligonucleotide
atggactggacctggagcatc

SEQ ID NO: 47 - DNA, 20 bp, Artificial Sequence
  Synthetic oligonucleotide
atggaattggggctgagctg

SEQ ID NO: 48 - DNA, 20 bp, Artificial Sequence
  Synthetic oligonucleotide
atggagtttggrctgagctg

SEQ ID NO: 49 - DNA, 21 bp, Artificial Sequence
  Synthetic oligonucleotide
atgaaacacctgtggttcttc

SEQ ID NO: 50 - DNA, 20 bp, Artificial Sequence
  Synthetic oligonucleotide
atggggtcaaccgccatcct

SEQ ID NO: 51 - DNA, 21 bp, Artificial Sequence
  Synthetic oligonucleotide
tgccagggggaagaccgatgg

SEQ ID NO: 52 - DNA, 33 bp, Artificial Sequence
  Synthetic oligonucleotide
ttcttggtggcagcagccacaggtgcccactcc

SEQ ID NO: 53 - DNA, 30 bp, Artificial Sequence
  Synthetic oligonucleotide
ctgctgaccatcccttcatgggtcttgtcc

SEQ ID NO: 54 - DNA, 33 bp, Artificial Sequence
  Synthetic oligonucleotide
ttccttgttgctattttaaaaggtgtccagtgt

SEQ ID NO: 55 - DNA, 27 bp, Artificial Sequence
  Synthetic oligonucleotide
gtggcagctcccagatgggtcctgtcc

SEQ ID NO: 56 - DNA, 33 bp, Artificial Sequence
  Synthetic oligonucleotide
ctcctcctgactgttctccaaggagtctgttcc

SEQ ID NO: 57 - DNA, 30 bp, Artificial Sequence
  Synthetic oligonucleotide
gtgctgggcctcccatggggtgtcctgtca

SEQ ID NO: 58 - DNA, 30 bp, Artificial Sequence
  Synthetic oligonucleotide
ttggtggcagcagcaacaggtgcccactcc

SEQ ID NO: 59 - DNA, 26 bp, Artificial Sequence
  Synthetic oligonucleotide
gaaggtgtgcacgccgctggtcagag

SEQ ID NO: 60 - DNA, 66 bp, Artificial Sequence
  Synthetic oligonucleotide
gtgcaattggtgcagtctggagcagaggtgaaaaagcccggggagtctctgaaaatctcctgtaag

SEQ ID NO: 61 - DNA, 30 bp, Artificial Sequence
  Synthetic oligonucleotide
gtgcaattggtggagtctgggggaggcttg

SEQ ID NO: 62 - DNA, 32 bp, Artificial Sequence
  Synthetic oligonucleotide
ctcgctgagctgacggtgaccagggttccctg

SEQ ID NO: 63 - DNA, 57 bp, Artificial Sequence
  Synthetic oligonucleotide
ccggaattcctcaccatggaaaccccagcgcagcttctcttcctcctgctactctgg

SEQ ID NO: 64 - DNA, 28 bp, Artificial Sequence
  Synthetic oligonucleotide
acccgtacgtttgatctccaccttggtc

SEQ ID NO: 65 - DNA, 45 bp, Artificial Sequence
  Synthetic oligonucleotide
ttacccaattgtgtcctgtcccaggtgcagctgcaggagtcgggc

SEQ ID NO: 66 - DNA, 40 bp, Artificial Sequence
  Synthetic oligonucleotide
tggaggctgagctgacggtgaccgtggtcccttggcccca

SEQ ID NO: 67 - DNA, 38 bp, Artificial Sequence
  Synthetic oligonucleotide
ttcccagggtcccgttccgacatccagatgacccagtc

SEQ ID NO: 68 - DNA, 31 bp, Artificial Sequence
  Synthetic oligonucleotide
agccaccgtacgtttgatatccactctggtc

SEQ ID NO: 69 - DNA, 26 bp, Artificial Sequence
  Synthetic oligonucleotide
tcggatccgctctggagaaggaggag

SEQ ID NO: 70 - DNA, 26 bp, Artificial Sequence
  Synthetic oligonucleotide
gcgaattcaagggcgtctccaaccgt

(19) United States
(12) Patent Application Publication
(10) Pub. No.: US 2003/0232387 A1
(43) Pub. Date: Dec. 18, 2003

(54) Antibodies that bind alphaE integrin

(75) Inventor: Chafen Lu, Newton, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Millennium Pharmaceuticals, Inc., Cambridge, MA

(21) Appl. No.: 10/173,551

(22) Filed: Jun. 14, 2002

(51) Int. Cl. G01N 33/53; A61K 39/395; C07K 16/28

(52) U.S. Cl. 435/7.1; 530/388.22; 424/143.1

(57) ABSTRACT

Antibodies and antigen-binding fragments of antibodies that bind αE integrin are disclosed. Some of the antibodies and antigen-binding fragments bind an activation induced epitope on integrin αE chain. In some embodiments, the antibodies are human. Nucleic acids and vectors encoding the antibodies or portions thereof, recombinant cells that contain the nucleic acids, and compositions comprising the antibodies or antigen-binding fragments are also disclosed. The invention also provides therapeutic and diagnostic methods that employ the antibodies and antigen-binding fragments.

### BACKGROUND OF THE INVENTION

Integrin receptors are important for regulating both lymphocyte recirculation and recruitment to sites of inflammation (Carlos, T. M. and Harlan, J. M., Blood 84:2068-2101 (1994)). The αE integrin αEβ7 is expressed on mucosal homing lymphocytes such as intestinal intraepithelial lymphocytes (IEL) and binds E-cadherin, which is expressed on epithelial cells, as well as a ligand on intestinal microvascular endothelial cell lines (Cepek, K. L. et al., Nature 372:190-193 (1994); Stauch U. G. et al., J. Immunol. 166:3506-3514 (2001)). As such, the αEβ7 integrin acts as a homing receptor that mediates lymphocyte migration to mucosal epithelium, such as intestinal epithelium (Schon, M. P. et al., J. Immunol. 162:6641-6649 (1999)).

αE integrins, like other integrins, can assume an activated or inactive conformation. Activated integrins bind ligand (e.g. E-cadherin) with high affinity. αE integrins, such as αEβ7, can be activated by divalent cations and/or by inside out signalling upon cellular stimulation with mitogens, growth factors and/or specific antigen (e.g., peptide/MHC).

Antibodies which bind αEβ7 integrin can interfere with αEβ7 integrin binding to its ligands (e.g., E-cadherin) and inhibit leukocyte migration to mucosal inflammatory sites (see, e.g., Ludviksson, B. R. et al., J. Immunol. 162:4975-4982 (1999); WO 00/30681 (Ludviksson, B. R. et al.)). However, a problem with using murine antibodies or other non-human antibodies for in vivo applications (e.g., diagnostic methods, therapeutic methods) in humans is that they are highly immunogenic and quickly induce a human anti-foreign antibody response (e.g., a human anti-mouse antibody response, HAMA). Such a human anti-foreign antibody response can result in rapid clearance of the foreign antibody and severely limit diagnostic or therapeutic uses or abrogate any therapeutic benefits.

Thus, a need exists for improved antibodies and antigen-binding fragments that can be used to diagnose and/or treat subjects having mucosal inflammatory disorders.

### SUMMARY OF THE INVENTION

The invention relates to antibodies and antigen-binding fragments of antibodies which bind an αE integrin (e.g., αEβ7 or other integrin comprising an αE chain). In one aspect, the invention is an antibody or antigen-binding fragment thereof that binds an activation-induced epitope on integrin αE chain, such as an epitope induced by exposure of an αE integrin to a divalent cation (e.g., Mn²⁺). For example, the activation-induced epitope can comprise amino acid residues in the I domain of integrin αE chain. In one embodiment, the antibody or antigen-binding fragment thereof binds an activation-induced epitope on human integrin αE chain. In another embodiment, the antibody or antigen-binding fragment thereof can inhibit the binding of a ligand (e.g., E-cadherin) to an αE integrin (e.g., αEβ7). In other embodiments, the antibody or antigen-binding fragment can inhibit αE integrin-mediated adhesion of a first cell expressing an αE integrin to a second cell bearing a ligand of an αE integrin, such as epithelial cells (e.g., intestinal epithelial cells) or endothelial cells. In particular embodiments, the antibody or antigen-binding fragment competitively inhibits binding of mAb 3G6 to αEβ7 integrin, or has the epitopic specificity of mAb 3G6.

In other embodiments, the antibody comprises one, two or three heavy chain complementarity determining regions (HCDR1, HCDR2 and/or HCDR3) having the amino acid sequences of the heavy chain CDRs of mAb 3G6 wherein, optionally, one or two amino acids in each heavy chain CDR can be conservatively substituted, and one, two or three light chain complementarity determining regions (LCDR1, LCDR2 and/or LCDR3) having the amino acid sequences of the light chain CDRs of mAb 3G6 wherein, optionally, one or two amino acids in each light chain CDR can be conservatively substituted. Preferably, the antibody comprises the three heavy chain CDRs and the three light chain CDRs of mAb 3G6. For example, in a particular embodiment the antibody can comprise the heavy chain variable region of mAb 3G6 (SEQ ID NO: 4) and the light chain variable region of mAb 3G6 (SEQ ID NO: 9).

In other embodiments, the antibody comprises one, two or three heavy chain complementarity determining regions (HCDR1, HCDR2 and/or HCDR3) having the amino acid sequences of the heavy chain CDRs of mAb 5E4 wherein, optionally, one or two amino acids in each heavy chain CDR can be conservatively substituted, and one, two or three light chain complementarity determining regions (LCDR1, LCDR2 and/or LCDR3) having the amino acid sequences of the light chain CDRs of mAb 5E4 wherein, optionally, one or two amino acids in each light chain CDR can be conservatively substituted. Preferably, the antibody comprises the three heavy chain CDRs and the three light chain CDRs of mAb 5E4. For example, in a particular embodiment the antibody can comprise the heavy chain variable region of mAb 5E4 (SEQ ID NO: 14) and the light chain variable region of mAb 5E4 (SEQ ID NO: 19).

In additional embodiments, the antibody comprises one, two or three heavy chain complementarity determining regions (HCDR1, HCDR2 and/or HCDR3) having the amino acid sequences of the heavy chain CDRs of mAb 8D5 wherein, optionally, one or two amino acids in each heavy chain CDR can be conservatively substituted, and one, two or three light chain complementarity determining regions (LCDR1, LCDR2 and/or LCDR3) having the amino acid sequences of the light chain CDRs of mAb 8D5 wherein, optionally, one or two amino acids in each light chain CDR can be conservatively substituted. Preferably, the antibody comprises the three heavy chain CDRs and the three light chain CDRs of mAb 8D5. For example, in a particular embodiment the antibody can comprise the heavy chain variable region of mAb 8D5 (SEQ ID NO: 24) and the light chain variable region of mAb 8D5 (SEQ ID NO: 29).

Preferred antibodies that bind an αE integrin (e.g., selectively bind an activation-induced epitope on integrin αE chain) include chimeric antibodies, humanized antibodies and antigen-binding fragments of the foregoing. Particularly preferred antibodies are of human origin. In specific embodiments, the invention is mAb 3G6, mAb 5E4 or mAb 8D5 or an antigen-binding fragment of mAb 3G6, mAb 5E4 or mAb 8D5.

The invention also relates to the heavy chains, light chains and portions of the heavy chains and light chains of the antibodies described herein. The invention also relates to fusion proteins comprising an antibody or portion thereof (e.g., heavy chain, light chain, variable region) of the invention and a non-immunoglobulin moiety. The invention also relates to immuno-conjugates comprising an antibody or antigen-binding fragment of the invention and a second moiety, such as a toxin (e.g., cytotoxin, cytotoxic agent), a therapeutic agent (e.g., a chemotherapeutic agent, an antimetabolite, an alkylating agent, an anthracycline, an antibiotic, an anti-mitotic agent, a biological response modifier (e.g., a cytokine (e.g., an interleukin, an interferon, a tumor necrosis factor), a growth factor (e.g., a neurotrophic factor)), a plasminogen activator, a radionuclide (e.g, a radioactive ion) or enzyme, for example.

The invention also relates to isolated and/or recombinant nucleic acids encoding the antibodies, antigen-binding fragments, heavy chains, light chains and portions of the heavy chains and light chains of the antibodies described herein, and to expression constructs or vectors comprising same. The invention also relates to a host cell that comprises a nucleic acid of the invention. In specific embodiments, the invention is hybridoma 3G6, hybridoma 5E4 or hybridoma 8D5.

The invention also relates to a method of treating a subject having an inflammatory disease or disorder comprising administering to said subject an effective amount of an antibody or antigen-binding fragment of the invention. In particular embodiments, the subject is a human. In other particular embodiments, the subject has an inflammatory bowel disease, such as ulcerative colitis or Crohn's disease.

The invention also relates to a method for detecting an activated αE integrin (e.g., activated αEβ7) comprising contacting a composition comprising an αE integrin with an antibody or antigen-binding fragment thereof which binds an activation-induced epitope on integrin αE chain and detecting formation of a complex between said antibody or antigen-binding fragment and said activated αE integrin.

The invention further relates to an antibody, antigen-binding fragment of an antibody, fusion protein or immuno-conjugate as described herein for use in therapy (including prophylaxis) or diagnosis, and to the use of an antibody, antigen-binding fragment of an antibody, fusion protein or immuno-conjugate of the invention for the manufacture of a medicament for the treatment of a particular disease or condition as described herein (e.g., a mucosal inflammatory disease (e.g., inflammatory bowel disease (e.g., ulerative colitis, Crohn's disease)), cancer (e.g., leukemia, lymphoma)).

### BRIEF DESCRIPTION OF THE DRAWINGS

FIGS. 1A-1H are fluorescent histograms showing binding of mAb 3G6 (IgG1) to transfected K562 cells that expressed an αEβ7 integrin under a variety of buffer conditions. The transfected cells were stained with isotype control antibody (human IgG1) in standard staining buffer (PBS/5% FBS) (FIG. 1A), with mAb 3G6 (IgG1) in standard staining buffer (FIG. 1B) or in buffer that contained EDTA (5 mM; FIG. 1C), in buffer that contained MnCl₂ (1 mM; FIG. 1D), in buffer that contained MgCl₂ (1 mM; FIG. 1E), in buffer that contained CaCl₂ (1 mM; FIG. 1F), in buffer that contained MgCl₂ and CaCl₂ (1 mM each; FIG. 1G) or in buffer that contained MgCl₂, CaCl₂ and MnCl₂ (1 mM each; FIG. 1H), and bound antibody was detected using a fluorescein isothiocyanate (FITC) labeled anti-human IgG antibody. The results show that binding of mAb 3G6 (IgG1) to the transfected cells was enhanced in buffer that contained Mn²⁺ (FIGS. 1D and 1H) and inhibited in buffer that contained EDTA (FIG. 1C) relative to binding in standard buffer.

FIG. 2A is an illustration of a nucleic acid sequence encoding the mature heavy chain variable region of mAb 3G6 (SEQ ID NO: 3) and the encoded amino acid sequence of the mature heavy chain variable region of mAb 3G6 (SEQ ID NO: 4). Complementarity determining region (CDR) 1 consists of amino acid residues 31-35 of SEQ ID NO: 4 (SEQ ID NO: 5), CDR 2 consists of amino acid residues 50-66 of SEQ ID NO: 4 (SEQ ID NO: 6), CDR 3 consists of amino acid residues 99-112 of SEQ ID NO: 4 (SEQ ID NO: 7).

FIG. 2B is an illustration of a nucleic acid sequence encoding the mature kappa light chain variable region of mAb 3G6 (SEQ ID NO: 8) and the encoded amino acid sequence of the mature light chain variable region of mAb 3G6 (SEQ ID NO: 9). Complementarity determining region (CDR) 1 consists of amino acid residues 24-34 of SEQ ID NO: 9 (SEQ ID NO: 10), CDR 2 consists of amino acid residues 50-56 of SEQ ID NO: 9 (SEQ ID NO: 11), CDR 3 consists of amino acid residues 89-98 of SEQ ID NO: 9 (SEQ ID NO: 12).

FIG. 3A is an illustration of a nucleic acid sequence encoding the mature heavy chain variable region of mAb 5E4 (SEQ ID NO:13) and the encoded amino acid sequence of the mature heavy chain variable region of mAb 5E4 (SEQ ID NO: 14). Complementarity determining region (CDR) 1 consists of amino acid residues 31-35 of SEQ ID NO: 14 (SEQ ID NO: 15), CDR 2 consists of amino acid residues 50-66 of SEQ ID NO: 14 (SEQ ID NO: 16), CDR 3 consists of amino acid residues 99-107 of SEQ ID NO: 14 (SEQ ID NO: 17).

FIG. 3B is an illustration of a nucleic acid sequence encoding the mature kappa light chain variable region of mAb 5E4 (SEQ ID NO: 18) and the encoded amino acid sequence of the mature light chain variable region of mAb 5E4 (SEQ ID NO: 19). Complementarity determining region (CDR) 1 consists of amino acid residues 24-34 of SEQ ID NO: 19 (SEQ ID NO: 20), CDR 2 consists of amino acid residues 50-56 of SEQ ID NO: 19 (SEQ ID NO: 21), CDR 3 consists of amino acid residues 89-98 of SEQ ID NO: 19 (SEQ ID NO: 22).

FIG. 4A is an illustration of a nucleic acid sequence encoding the mature heavy chain variable region of mAb 8D5 (SEQ ID NO: 23) and the encoded amino acid sequence of the mature heavy chain variable region of mAb 8D5 (SEQ ID NO: 24). Complementarity determining region (CDR) 1 consists of amino acid residues 31-35 of SEQ ID NO: 24 (SEQ ID NO: 25), CDR 2 consists of amino acid residues 50-65 of SEQ ID NO: 24 (SEQ ID NO: 26), CDR 3 consists of amino acid residues 98-117 of SEQ ID NO: 24 (SEQ ID NO: 27).

FIG. 4B is an illustration of a nucleic acid sequence encoding the mature kappa light chain variable region of mAb 8D5 (SEQ ID NO: 28) and the encoded amino acid sequence of the mature light chain variable region of mAb 8D5 (SEQ ID NO: 29). Complementarity determining region (CDR) 1 consists of amino acid residues 24-34 of SEQ ID NO: 29 (SEQ ID NO: 30), CDR 2 consists of amino acid residues 50-56 of SEQ ID NO: 29 (SEQ ID NO: 31), CDR 3 consists of amino acid residues 89-97 of SEQ ID NO: 29 (SEQ ID NO: 32).

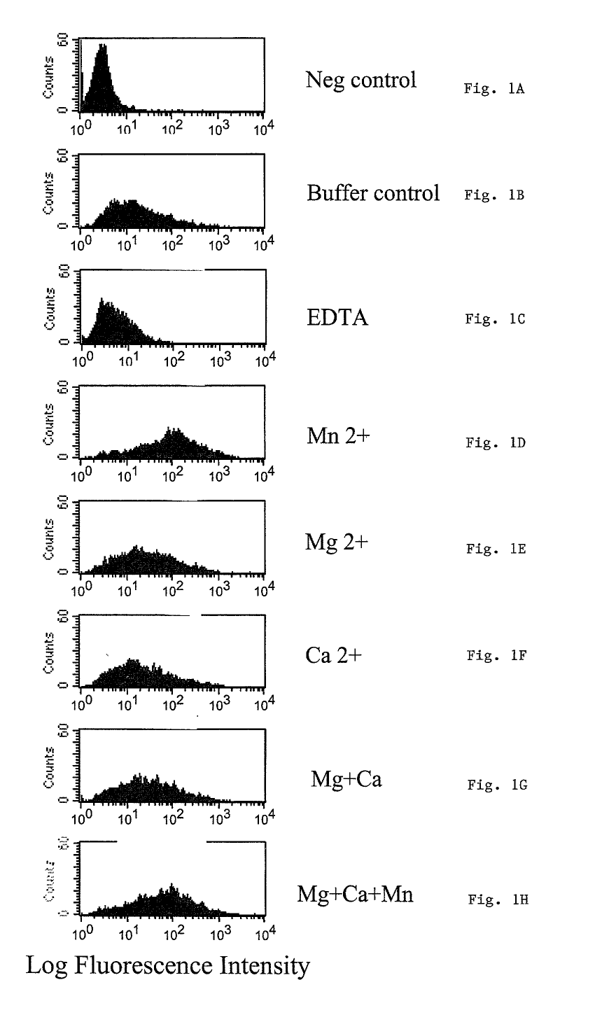

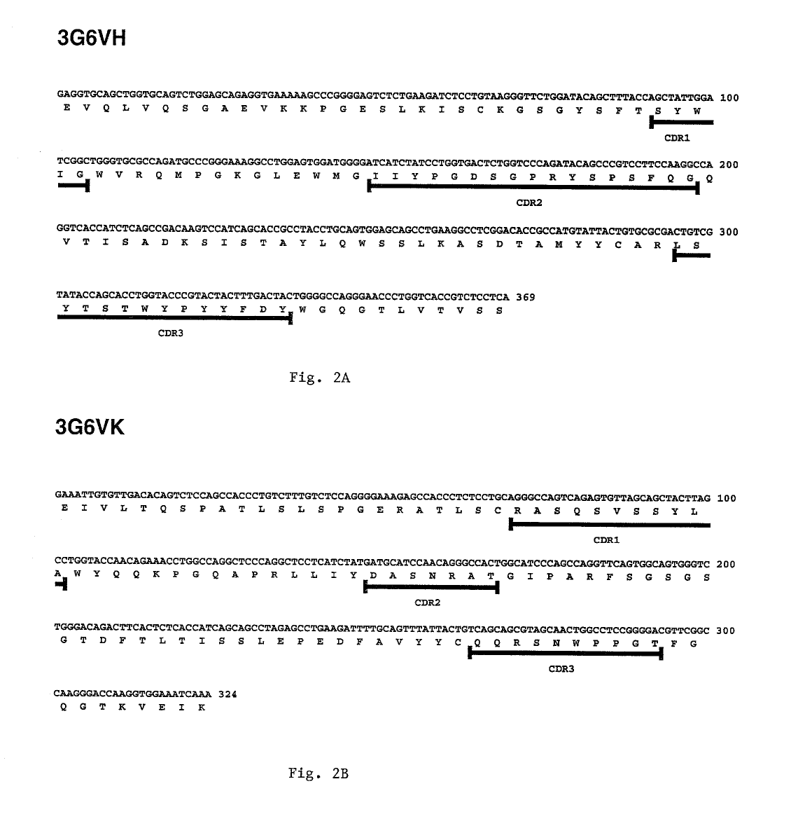

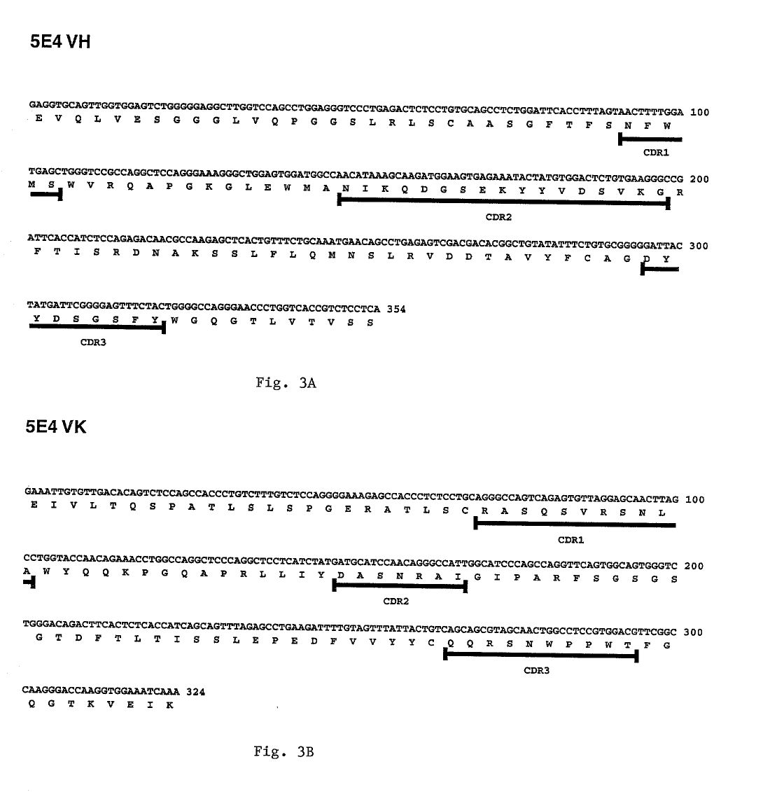

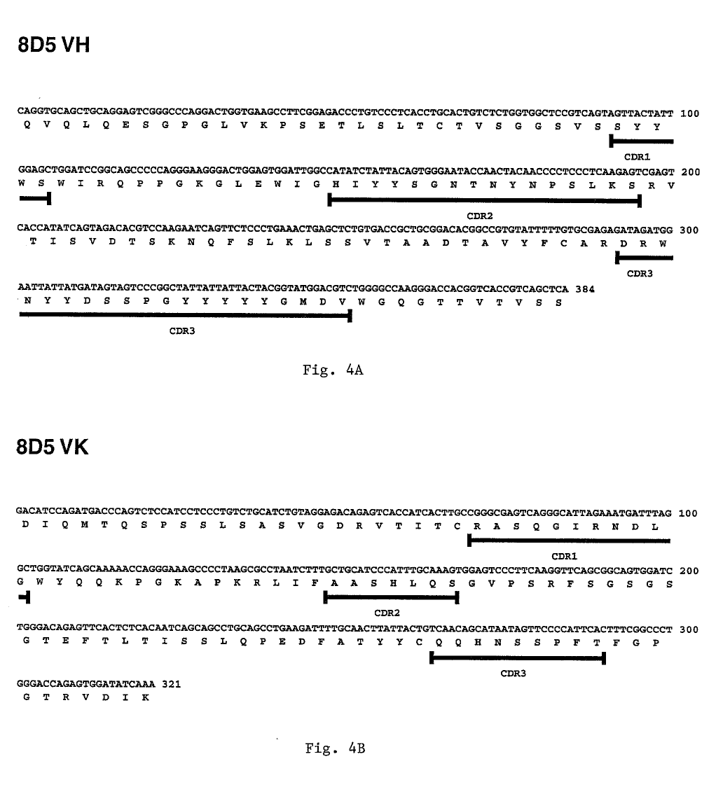

### DETAILED DESCRIPTION OF THE INVENTION

As used herein, “activation-induced epitope” refers to an epitope that is present on an activated αE integrin (e.g., integrin αE chain (CD103), an αEβ7 integrin) but not on non-activated αE integrin. An activated αE integrin is an αE integrin that binds ligand (e.g., E-cadherin) with high affinity, while a non-activated αE integrin binds the ligand with low affinity. (See, Higgins, J. M. G. et al., J. Biol. Chem. 140:197-210 (1998).) An αE integrin can be activated, for example, by exposure to divalent cations (e.g., Mn²⁺). When the αE integrin is expressed on the surface of a cell, it can be activated upon exposure of the cell to phorbol esters (e.g., Phorbol 12-myristate 13-acetate (PMA)), or to suitable growth factors and/or mitogens (e.g., concanavalin A). An αE integrin expressed on a T cell can be activated by signals transduced through the T cell receptor (TCR) complex (e.g., upon TCR binding to specific MHC-peptide complexes, crosslinking with anti-CD3 antibody).

An antibody that “binds an activation-induced epitope” on integrin αE chain binds integrin αE chain under activation conditions (e.g., in the presence of divalent cations (e.g., Mn²⁺)) but does not significantly bind in the absence of activation (e.g., when a suitable chelating agent (e.g., Ethylenediaminetetraacetic acid (EDTA)) is present).

As used herein, an antibody and antigen-binding fragment thereof that “binds” an αE integrin (e.g., an activated αE integrin, an αEβ7 integrin, an integrin αE chain (CD103)) has binding specificity for the αE integrin. The terms “binding specificity” or “specific” when referring to an antibody-antigen interaction indicate that the antibody can discriminate between one or more αE integrins (e.g., an activated αE integrin, an αEβ7 integrin, an integrin αE chain (CD103)) and other antigens, rather than to indicate that the antibody can bind only one antigen. For example, in certain embodiments, the antibody or antigen-binding fragments of the invention can “selectively bind” an αE integrin. Such selective antibodies or antigen-binding fragments may bind another antigen with low affinity, but bind said αE integrin with higher affinity. Under appropriate binding conditions (e.g., physiological conditions), an antibody or antigen-binding fragment thereof that selectively binds an αE integrin will bind the αE integrin but will not significantly bind other antigens. An antibody or antigen-binding fragment of an antibody does not “significantly bind” an antigen when the extent of binding is less than about 25%, preferably less than about 15%, more preferably less than about 10%, most preferably less than about 5% or less than about 2% or 1% of the level of binding to an antigen that is “selectively” bound under the same conditions (e.g., physiological conditions). The concentration of antibody and other conditions required to provide selectivity for an αE integrin (e.g., an antibody concentration and pH which reduces or eliminates non-selective binding) can be readily determined using any suitable method, such as titration.

As used herein, the term “functionally rearranged” refers to a segment of DNA from an immunoglobulin locus which has undergone V(D)J recombination, with or without insertion or deletion of nucleotide(s) (e.g., N nucleotides, P nucleotides) and/or somatic mutation, thereby producing an immunoglobulin gene which encodes an immunoglobulin variable region or immunoglobulin chain (e.g., heavy chain, light chain). A functionally rearranged immunoglobulin gene can be directly or indirectly identified using suitable methods, such as, for example, nucleotide sequencing, hybridization (e.g., Southern blotting, Northern blotting) using probes which can anneal to coding joints between gene segments (e.g., VH, VL, D, JH, JL) or enzymatic amplification of immunoglobulin genes (e.g., polymerase chain reaction) with primers which can anneal to coding joints between gene segments. Whether a cell produces an antibody comprising a particular variable region or a variable region comprising a particular sequence (e.g., a CDR sequence) can also be determined using suitable methods. In one example, mRNA can be isolated from an antibody producing cell (e.g., a hybridoma) and used to produce cDNA. The cDNA can be cloned and sequenced or can be amplified (e.g., by polymerase chain reaction) using a first primer which anneals specifically to a portion of the variable region of interest (e.g., CDR, coding joint) and a second primer which anneals specifically to non-variable region sequences (e.g., C_{H}1, C_{L}).

As used herein, the phrase “of human origin” refers to antibodies, antigen-binding fragments of antibodies and portions or regions of antibodies (e.g., variable regions, complementarity determining regions (CDRs), framework regions (FRs), constant regions) having amino acid sequences that are encoded by nucleotide sequences derived from human (Homo sapiens) germ line immunoglobulin genes. For example, an antibody of human origin can be encoded by human germ line immunoglobulin genes that have been functionally rearranged to produce a functional gene that can be expressed to produce an antibody. As described herein, functionally rearranged genes that encode an antibody chain can include sequences that are not found in the germ line, such as N nucleotides and P nucleotides, and mutations that can occur as part of the processes that produce high-affinity antibodies (e.g., somatic mutation, affinity maturation, clonal selection). Functionally rearranged immunoglobulin genes of human origin, including those that include non-germ line sequences, can be generated via natural processes in a suitable in vivo expression system (e.g., a human, a human-antibody transgenic animal), artificially using any suitable methods (e.g., recombinant DNA technology, phage display) or any combination of natural and artificial processes. Antibodies, antigen-binding fragments of antibodies and portions or regions of antibodies of human origin can be produced, for example, by expression of a nucleic acid of non-human origin (e.g., a synthetic nucleic acid) that has the requisite nucleotide sequence.

An antibody, antigen-binding fragment of an antibody or a portion of an antibody (e.g., a framework region) “of human origin” can have an amino acid sequence that is encoded by a nucleic acid that has a nucleotide sequence that is a consensus of the nucleotide sequences of a number of naturally occurring human antibody genes or human germ line sequences, or have an amino acid sequence that is a consensus of the amino acid sequences of a number of naturally occurring human antibodies or amino acid sequences encoded in the human germ line. A number of human antibody consensus sequences are available, including consensus sequences for the different subgroups of human variable regions (see, Kabat, E. A., et al., Sequences of Proteins of Immunological Interest, Fifth Edition, U.S. Department of Health and Human Services, U.S. Government Printing Office (1991). The Kabat database and its applications are freely available on line. (See, Johnson, G. and Wu, T. T., Nucleic Acids Research 29:205-206 (2001).)

As used herein, the phrase “human antibody” refers to antibodies or antigen-binding fragments of antibodies in which the variable and constant regions (if present) have amino acid sequences that are encoded by nucleotide sequences derived from human (Homo sapiens) germline immunoglobulin genes. A “human antibody” can include sequences that are not encoded in the germline (e.g., due to N nucleotides, P nucleotides, and mutations that can occur as part of the processes that produce high-affinity antibodies such as, somatic mutation, affinity maturation, clonal selection)) that occur as a result of biological processes in a suitable in vivo expression system (e.g., a human, a human-antibody transgenic animal). Antibodies, antigen-binding fragments of antibodies and portions or regions of human antibodies can be produced, for example, by expression of a nucleic acid of non-human origin (e.g., a synthetic nucleic acid) that has the requisite nucleotide sequence.

As used herein, the phrase “CDR-grafted” antibody refers to antibodies and antigen-binding fragments of antibodies that comprise a CDR that is not naturally associated with the framework regions of the antibody or antigen-binding fragment. Generally the CDR is from an antibody from a first species and the framework regions and constant regions (if present) are from an antibody from a different species. The CDR-grafted antibody can be a “humanized antibody.”

As used herein, “humanized antibody” refers to an antibody or antigen-binding fragment thereof comprising a CDR that is not of human origin and framework and/or constant regions that are of human origin. For example, a humanized antibody can comprise a CDR derived from an antibody of nonhuman origin (e.g., natural antibody such as a murine (e.g., mouse, rat) antibody, artificial antibody) that binds an αE integrin, preferably integrin αE chain (CD103), and framework and constant regions (if present) of human origin (e.g., a human framework region, a human consensus framework region, a human constant region (e.g., CL, CH1, hinge, CH2, CH3, CH4)). CDR-grafted single chain antibodies containing a CDR of non-human origin and framework and constant regions (if present) of human origin (e.g., CDR-grafted scFV) are also encompassed by the term humanized antibody.

As used herein, the term “chimeric antibody” refers to an antibody or antigen-binding fragment thereof comprising a variable region from an antibody from a first species and a constant region from an antibody from a different species. None of the portions which comprise a chimeric antibody need to be of human origin. For example, a chimeric antibody can comprise a variable region from a rodent (e.g., mouse) antibody and a constant region of a non-human primate antibody (e.g., a chimpanzee constant region).

The antibody of the invention can be a single chain antibody (e.g., a single chain Fv (scFv)) and can include a linker moiety (e.g., a linker peptide) not found in native antibodies. For example, an scFv can comprise a linker peptide, such as two to about twenty glycine residues or other suitable linker, which connects a heavy chain variable region to a light chain variable region. For the purposes of the invention, the presence of such a linker does not affect the status of the single chain antibody as being “of human origin” or “human.” For example, a human scFv can comprise a human heavy chain variable region and a human light chain variable region that are connected through a suitable peptide linker.

“Conservative amino acid substitution” refers to the replacement of a first amino acid by a second amino acid that has chemical and/or physical properties (e.g., charge, structure, polarity, hydrophobicity/hydrophilicity) which are similar to those of the first amino acid. For example, replacement of one amino acid by another within the following groups is a conservative amino acid substitution: Ala, Val, Leu, and Ile; Ser and Thr; Asp and Glu; Asn and Gln; Lys and Arg; Phe and Tyr.

A nucleotide sequence encoding a human (Homo sapiens) integrin αE chain (CD103), used in the studies described herein and deposited in GenBank under accession number L25851, is presented as SEQ ID NO: 1. (See also, Shaw et al., J. Biol. Chem. 269:6016-6025 (1994).) The nucleotide sequence has an open-reading frame beginning at position 126. The amino acid sequence of a human integrin αE chain encoded by SEQ ID NO: 1 is presented as SEQ ID NO: 2. The human integrin αE chain contains a signal peptide (amino acid residues −18 to −1 of SEQ ID NO: 2), an X-domain (amino acid residues 126-180 of SEQ ID NO: 2) and an I-Domain (residues 181-372 of SEQ ID NO: 2). The entire teachings of GenBank Accession No. L25851 are incorporated herein by reference.

A nucleotide sequence encoding a human (Homo sapiens) E-cadherin used in the studies described herein and deposited in GenBank under accession number L08599 is presented as SEQ ID NO:33. The nucleotide sequence has an open-reading frame beginning at position 109. The amino acid sequence of a human E-cadherin encoded by SEQ ID NO: 33 is presented as SEQ ID NO: 34. The entire teachings of GenBank Accession No. L08599 are incorporated herein by reference.

A nucleotide sequence encoding a human (Homo sapiens) integrin α4 chain used in the studies described herein and deposited in GenBank under accession number L12002 is presented as SEQ ID NO: 35. The nucleotide sequence has an open-reading frame beginning at position 411. The amino acid sequence of an integrin α4 chain encoded by SEQ ID NO: 35 is presented as SEQ ID NO:36. The entire teachings of GenBank Accession No. L12002 are incorporated herein by reference.

A nucleotide sequence encoding a human (Homo sapiens) integrin β7 chain used in the studies described herein and deposited in GenBank under accession number M62880 is presented as SEQ ID NO:37. The nucleotide sequence has an open-reading frame beginning at position 114. The amino acid sequence of an integrin β7 chain encoded by SEQ ID NO: 37 is presented as SEQ ID NO: 38. The entire teachings of GenBank Accession No. M62880 are incorporated herein by reference.

Antibodies and Antibody Producing Cells

The antibody of the invention can be polyclonal or monoclonal, and the term “antibody” is intended to encompass both polyclonal and monoclonal antibodies. The terms polyclonal and monoclonal refer to the degree of homogeneity of an antibody preparation, and are not intended to be limited to particular methods of production. The term “antibody” as used herein encompasses antigen-binding fragments of antibodies, including antigen-binding fragments of human, humanized, chimeric, CDR-grafted, veneered or single-chain antibodies.

Antibodies which bind an αE integrin can be selected from a suitable collection of natural or artificial antibodies or raised against an appropriate immunogen in a suitable host. For example, antibodies can be raised by immunizing a suitable host (e.g., mouse, human antibody-transgenic mouse) with a suitable immunogen, such as an isolated or purified αE integrin (e.g., αEβ7) or cells expressing a recombinant αE integrin (e.g., cell that expresses an exogenous nucleic acid encoding human integrin αE chain (CD103)). In addition, cells expressing a recombinant αE integrin, such as transfected cells, can be used in a screen for antibody which binds thereto (See e.g., Chuntharapai et al., J. Immunol., 152: 1783-1789 (1994); Chuntharapai et al., U.S. Pat. No. 5,440,021).

Preparation of immunizing antigen, and polyclonal and monoclonal antibody production can be performed using any suitable technique. A variety of methods have been described. (See, e.g., Kohler et al., Nature, 256: 495-497 (1975) and Eur. J. Immunol. 6: 511-519 (1976); Milstein et al., Nature 266: 550-552 (1977); Koprowski et al., U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol. 2 (Supplement 27, Summer '94), Ausubel, F. M. et al., Eds., (John Wiley & Sons: New York, N.Y.), Chapter 11, (1991).) Generally, where a monoclonal antibody is desired, a hybridoma is produced by fusing a suitable immortal cell line (e.g., a myeloma cell line such as SP2/0, P3X63Ag8.653 or a heteromyeloma) with antibody-producing cells. Antibody-producing cells can be obtained from the peripheral blood or, preferably the spleen or lymph nodes, of humans, human-antibody transgenic animals or other suitable animals immunized with the antigen of interest. Cells that produce antibodies of human origin (e.g., a human antibody) can be produced using suitable methods, for example, fusion of a human antibody-producing cell and a heteromyeloma or trioma, or immortalization of an activated human B cell via infection with Epstein Barr virus. (See, e.g., U.S. Pat. No. 6,197,582 (Trakht); Niedbala et al., Hybridoma, 17:299-304 (1998); Zanella et al., J Immunol Methods, 156:205-215 (1992); Gustafsson et al., Hum Antibodies Hybridomas, 2:26-32 (1991).) The fused or immortalized antibody-producing cells (hybridomas) can be isolated using selective culture conditions, and cloned by limiting dilution. Cells which produce antibodies with the desired specificity can be identified using a suitable assay (e.g., ELISA).

Other suitable methods of producing or isolating antibodies or antigen-binding fragments of the desired specificity can be used, including, for example, methods which select a recombinant antibody or antigen-binding fragment thereof from a library, such as a phage display library. Such libraries can contain antibodies or antigen-binding fragments of antibodies that contain natural or artificial amino acid sequences. For example, the library can contain Fab fragments which contain artificial CDRs (e.g., random amino acid sequences) and human framework regions. (See, for example, U.S. Pat. No. 6,300,064 (Knappik, et al.), the entire teachings of which are incorporated herein by reference.)

Human antibodies and nucleic acids encoding same can be obtained from a human or from human-antibody transgenic animals. Human-antibody transgenic animals (e.g., mice) are animals that are capable of producing a repertoire of human antibodies, such as XENOMOUSE (Abgenix, Fremont, Calif.), HUMAB-MOUSE, KIRIN TC MOUSE or KM-MOUSE (MEDAREX, Princeton, N.J.). Generally, the genome of human-antibody transgenic animals has been altered to include a transgene comprising DNA from a human immunoglobulin locus that can undergo functional rearrangement. An endogenous immunoglobulin locus in a human-antibody transgenic animal can be disrupted or deleted to eliminate the capacity of the animal to produce antibodies encoded by an endogenous gene. Suitable methods for producing human-antibody transgenic animals are well known in the art. (See, for example, U.S. Pat. Nos. 5,939,598 and 6,075,181 (Kucherlapati et al.), U.S. Pat. Nos. 5,569,825, 5,545,806, 5,625,126, 5,633,425, 5,661,016, and 5,789,650 (Lonberg et al.), Jakobovits et al., Proc. Natl. Acad. Sci. USA, 90: 2551-2555 (1993), Jakobovits et al., Nature, 362: 255-258 (1993), Jakobovits et al. WO 98/50433, Jakobovits et al. WO 98/24893, Lonberg et al. WO 98/24884, Lonberg et al. WO 97/13852, Lonberg et al. WO 94/25585, Lonberg et al. EP 0 814 259 A2, Lonberg et al. GB 2 272 440 A, Lonberg et al., Nature 368:856-859 (1994), Lonberg et al., Int Rev Immunol 13(1):65-93 (1995), Kucherlapati et al. WO 96/34096, Kucherlapati et al. EP 0 463 151 B1, Kucherlapati et al. EP 0 710 719 A1, Surani et al. U.S. Pat. No. 5,545,807, Bruggemann et al. WO 90/04036, Bruggemann et al. EP 0 438 474 B1, Taylor et al., Int. Immunol. 6(4)579-591 (1994), Taylor et al., Nucleic Acids Research 20(23):6287-6295 (1992), Green et al, Nature Genetics 7:13-21 (1994), Mendez et al., Nature Genetics 15:146-156 (1997), Tuaillon et al., Proc Natl Acad Sci USA 90(8)3720-3724 (1993) and Fishwild et al., Nat Biotechnol 14(7):845-851 (1996), the teachings of each of the foregoing are incorporated herein by reference in their entirety.)

As described herein, human-antibody transgenic animals can be immunized with a suitable composition comprising an antigen of interest (e.g., a recombinant cell expressing an αEβ7 integrin). Antibody producing cells can be isolated and fused to form hybridomas using conventional methods. Hybridomas that produce human antibodies having the desired characteristics (e.g., specificity, affinity) can be identified using any suitable assay (e.g, ELISA) and, if desired, selected and subcloned using suitable culture techniques.

Human-antibody transgenic animals provide a source of nucleic acids that can be enriched in nucleic acids that encode antibodies having desired properties, such as specificity and affinity. For example, nucleic acids encoding antibodies or antibody variable regions can be isolated from human-antibody transgenic mice that have been immunized with an αE integrin. The isolated nucleic acids or portions thereof (e.g., portions encoding variable regions, CDRs, framework regions) can be expressed using any suitable method (e.g., phage display) to produce a library of antibodies or antigen-binding fragments of antibodies (e.g., single chain antigen-binding fragments, double chain antigen-binding fragments) that is enriched for antibodies or antigen-binding fragments that bind αE. Such a library can exhibit enhanced diversity (e.g., combinatorial diversity through pairing of heavy chain variable regions and light chain variable regions) relative to the repertoire of antibodies produced in the immunized human-antibody transgenic animal. The library can be screened using any suitable assay (e.g., an αE binding assay) to identify antibodies or antigen-binding fragments having desired properties (e.g., specificity, affinity). The nucleic acids encoding antibody or antigen-binding fragments having desired properties can be recovered using any suitable methods. (See, e.g., U.S. Pat. No. 5,871,907 (Winter et al.) and U.S. Pat. No. 6,057,098 (Buechler et al.), the entire teachings of each of the foregoing are incorporated herein by reference.)

The antibody of the invention can be a CDR-grafted (e.g., humanized) antibody or an antigen-binding fragment thereof. The CDRs of a CDR-grafted antibody can be derived from a suitable antibody which binds an αE integrin (referred to as a donor antibody). For example, suitable CDRs can be derived from mAb 3G6, mAb 5E4 or mAb 8D5 which, as described herein, bind integrin αE chain (CD103) or from any other suitable antibody. Other sources of suitable CDRs include natural and artificial αE integrin-specific antibodies obtained from nonhuman sources, such as rodent (e.g., mouse, rat), rabbit, pig, goat, non-human primate (e.g., monkey) or non-human library.

The framework regions of a CDR-grafted antibody are preferably of human origin, and can be derived from any human antibody variable region having sequence similarity to the analogous or equivalent region (e.g., light chain variable region) of the antigen binding region of the donor antibody. Other sources of framework regions of human origin include human variable region consensus sequences. (See, e.g., Kettleborough, C. A. et al., Protein Engineering 4:773-783 (1991); Carter et al., WO 94/04679; Kabat, E. A., et al., Sequences of Proteins of Immunological Interest, Fifth Edition, U.S. Department of Health and Human Services, U.S. Government Printing Office (1991)).

In one embodiment, the framework regions of a CDR-grafted (e.g., humanized) antibody chain can be derived from a variable region of human origin having at least about 65% overall amino acid sequence identity, and preferably at least about 70% overall amino acid sequence identity, with the amino acid sequence of the variable region of the donor antibody. A suitable framework region can also be derived from a antibody of human origin having at least about 65% amino acid sequence identity, and preferably at least about 70%, 80%, 90% or 95% amino acid sequence identity over the length of the framework region within the amino acid sequence of the equivalent portion (e.g., framework region) of the donor antibody. For example, a suitable framework region of human origin can be derived from an antibody of human origin (e.g., a human antibody) having at least about 65% amino acid sequence identity, and preferably at least about 70%, 80%, 90% or 95% amino acid sequence identity, over the length of the particular framework region being used, when compared to the amino acid sequence of the equivalent portion (e.g., framework region) of the donor antibody. Amino acid sequence identity can be determined using a suitable amino acid sequence alignent algorithm, such as CLUSTAL W, using the default parameters. (Thompson J. D. et al., Nucleic Acids Res. 22:4673-4680 (1994).)

Framework regions of human origin can include amino acid substitutions or replacements, such as “back mutations” which replace an amino acid residue in the framework region of human origin with a residue from the corresponding position of the donor antibody. One or more mutations in the framework region can be made, including deletions, insertions and substitutions of one or more amino acids. Preferably, the CDR-grafted (e.g., humanized) antibody binds αE integrin with an affinity similar to, substantially the same as, or better than that of the donor antibody. Variants can be produced by a variety of suitable methods, including mutagenesis of nonhuman donor or acceptor human chains. (See, e.g., U.S. Pat. Nos. 5,693,762 (Queen et al.) and 5,859,205 (Adair et al.), the entire teachings of which are incorporated herein by reference.)

Constant regions of antibodies, antibody chains (e.g, heavy chain, light chain) or fragments or portions thereof of the invention, if present, can be derived from any suitable source. For example, constant regions of human, humanized and certain chimeric antibodies, antibody chains (e.g, heavy chain, light chain) or fragments or portions thereof, if present can be of human origin and can be derived from any suitable human antibody or antibody chain. For example, a constant region of human origin or portion thereof can be derived from a human κ or λ light chain, and/or a human γ (e.g., γ1, γ2, γ3, γ4), μ, α (e.g., α1, α2), δ or ε heavy chain, including allelic variants. In certain embodiments, the antibody or antigen-binding fragment (e.g., antibody of human origin, human antibody) can include amino acid substitutions or replacements that alter or tailor function (e.g., effector function). For example, a constant region of human origin (e.g., γ1 constant region, γ2 constant region) can be designed to reduce complement activation and/or Fc receptor binding. (See, for example, U.S. Pat. Nos. 5,648,260 (Winter et al.), 5,624,821 (Winter et al.) and 5,834,597 (Tso et al.), the entire teachings of which are incorporated herein by reference.) Preferably, the amino acid sequence of a constant region of human origin that contains such amino acid substitutions or replacements is at least about 95% identical over the full length to the amino acid sequence of the unaltered constant region of human origin, more preferably at least about 99% identical over the full length to the amino acid sequence of the unaltered constant region of human origin.

Humanized antibodies or antigen-binding fragments of a humanized antibody can be prepared using any suitable method. Several such methods are well-known in the art. (See, e.g., U.S. Pat. No. 5,225,539 (Winter), U.S. Pat. No. 5,530,101 (Queen et al.).) The portions of a humanized antibody (e.g., CDRs, framework, constant region) can be obtained or derived directly from suitable antibodies (e.g., by de novo synthesis of a portion), or nucleic acids encoding an antibody or chain thereof having the desired property (e.g., binds αE integrin) can be produced and expressed. Humanized immunoglobulins comprising the desired portions (e.g., CDR, FR, constant region) of human and nonhuman origin can be produced using synthetic and/or recombinant nucleic acids to prepare a nucleic acid (e.g., cDNA) encoding the desired humanized chain. To prepare a portion of a chain, one or more stop codons can be introduced at the desired position. For example, nucleic acid (e.g., DNA) sequences coding for newly designed humanized variable regions can be constructed using PCR mutagenesis methods to alter existing DNA sequences. (See, e.g., Kamman, M., et al., Nucl. Acids Res. 17:5404 (1989).) PCR primers coding for the new CDRs can be hybridized to a DNA template of a previously humanized variable region which is based on the same, or a very similar, human variable region (Sato, K., et al., Cancer Research 53:851-856 (1993)). If a similar DNA sequence is not available for use as a template, a nucleic acid comprising a sequence encoding a variable region sequence can be constructed from synthetic oligonucleotides (see e.g., Kolbinger, F., Protein Engineering 8:971-980 (1993)). A sequence encoding a signal peptide can also be incorporated into the nucleic acid (e.g., on synthesis, upon insertion into a vector). The natural signal peptide sequence from the acceptor antibody, a signal peptide sequence from another antibody or other suitable sequence can be used (see, e.g., Kettleborough, C. A., Protein Engineering 4:773-783 (1991)). Using these methods, methods described herein or other suitable methods, variants can be readily produced. In one embodiment, cloned variable regions can be mutated, and sequences encoding variants with the desired specificity can be selected (e.g., from a phage library; see, e.g., U.S. Pat. No. 5,514,548 (Krebber et al.) and WO 93/06213 (Hoogenboom et al.)).

The antibody of the invention can be a chimeric antibody or an antigen-binding fragment of a chimeric antibody. Preferably, the chimeric antibody or antigen-binding fragment thereof comprises a variable region of non-human origin and a constant region of human origin (e.g., a human constant region).

Chimeric antibodies and antigen-binding fragments of chimeric antibodies that bind αE integrin can be prepared using any suitable method. Several suitable methods are well-known in the art. (See, e.g., U.S. Pat. No. 4,816,567 (Cabilly et al.), U.S. Pat. No. 5,116,946 (Capon et al.).) Generally, chimeric antibodies are produced by preparing, for each of the light and heavy chain components of the chimeric immunoglobulin, a recombinant nucleic acid comprising a first nucleotide sequence encoding at least the variable region of an antibody from a first species that binds αE integrin that is joined in frame to a second nucleotide sequence encoding at least a part of a constant region from an antibody of a different species. Generally, the recombinant nucleic acid encodes a chimeric heavy chain or a chimeric light chain. However, if desired, a single recombinant nucleic acid encoding a chimeric heavy chain and a chimeric light chain can be prepared. The recombinant nucleic acids can be assembled in or inserted into an expression vector. The recombinant nucleic acid(s) can be introduced into a suitable host cell that is capable of expressing the chimeric antibody or chimeric antibody chain using any suitable method (e.g., transfection, transformation, infection) to produce a recombinant host cell. The recombinant host cell can be maintained under conditions suitable for expression of the chimeric antibody or chimeric antibody chain and the antibody or chain can be recovered.

Nucleic acids encoding the variable region of antibody light and heavy chains can be obtained from cells (e.g., B cells, hybridoma cells) that produce an antibody that binds αE integrin. For example, nucleic acids that encode human heavy and light chain variable regions that can bind αE integrin can be obtained from hybridomas 3G6, 5E4 and 8D5, and from recombinant cell lines CHO 3G6 C1.2D6 and CHO 5E4 A1.2C12, described herein. Nucleic acids that encode constant regions can be obtained from suitable sources using any suitable technique, such a conventional techniques of recombinant DNA technology. The nucleotide sequences of nucleic acids encoding human κ or λ light chain constant regions, and γ (e.g., γ1, γ2, γ3, γ4), μ, α (e.g., α1, α2), δ or ε human heavy chain constant regions are readily available.

The invention also relates to a bispecific antibody or antigen-binding fragment thereof (e.g., F(ab′)₂), which binds an αE integrin and at least one other antigen. In a particular embodiment, the bispecific antibody, or antigen-binding fragment thereof binds an activation-induced epitope on an αE integrin (e.g., integrin αE chain (CD103)). In other embodiments, the bispecific antibody or antigen-binding fragment thereof has the epitopic specificity of mAb 3G6, mAb 5E4 or mAb 8D5 and at least one other antibody. Bispecific antibodies can be secreted by triomas and hybrid hybridomas. Generally, triomas are formed by fusion of a hybridoma and a lymphocyte (e.g., antibody secreting B cell) and hybrid hybridomas are formed by fusion of two hybridomas. Each of the cells that are fused to produce a trioma or hybrid hybridoma produces a monospecific antibody. However, triomas and hybrid hybridomas can produce an antibody containing antigen binding sites which recognize different antigens. The supernatants of triomas and hybrid hybridomas can be assayed for bispecific antibody using a suitable assay (e.g., ELISA), and bispecific antibodies can be purified using conventional methods. (See, e.g., U.S. Pat. No. 5,959,084 (Ring et al.) U.S. Pat. No. 5,141,736 (Iwasa et al.), U.S. Pat. Nos. 4,444,878, 5,292,668 and 5,523,210 (Paulus et al.) and U.S. Pat. No. 5,496,549 (Yamazaki et al.).)

The various portions of an antibody (e.g., mouse antibody, human antibody, humanized antibody, chimeric antibody and antigen-binding fragments of the foregoing) can be joined together chemically using conventional techniques, or can be prepared as a continuous polypeptide chain by expression (in vivo or in vitro) of a nucleic acid (one or more nucleic acids) encoding antibody. For example, nucleic acids encoding a human, humanized or chimeric chain can be expressed in vivo or in vitro to produce a continuous polypeptide chain. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0 451 216 B1; and Padlan, E. A. et al., EP 0 519 596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single chain antibodies.

The invention also relates to antigen-binding fragments of antibodies that retain the capacity to bind antigen (e.g., an αE integrin, an activation-induced epitope on integrin αE chain). Such antigen-binding fragments of antibodies retain the antigen binding function of a corresponding full-length antibody (e.g., binding specificity for an αE integrin), and preferably inhibit binding of ligand (e.g., E-cadherin) to an αE integrin (e.g., αEβ7). Antigen-binding fragments of antibodies encompassed by the invention include, Fv fragments (e.g., single chain Fv fragments (scFv)), Fab fragments, Fab′ fragments and F(ab′)₂ fragments, for example. Such antigen-binding fragments can be produced using any suitable method, for example by enzymatic cleavage and/or using recombinant DNA technology. For example, an antibody can be cleaved with papain or pepsin to yield a Fab fragment or F(ab′)₂ fragment, respectively. Other proteases with the requisite substrate specificity can also be used to generate antigen-binding fragments of antibodies, such as Fab fragments or F(ab′)₂ fragments. Similarly, Fv fragments can be prepared by digesting an antibody with a suitable protease or using recombinant DNA technology. For example, a nucleic acid can be prepared that encodes a light chain variable region and heavy chain variable region that are connected by a suitable peptide linker, such as a chain of two to about twenty Glycyl residues. The nucleic acid can be introduced into a suitable host (e.g., E. coli) using any suitable technique (e.g., transfection, transformation, infection), and the host can be maintained under conditions suitable for expression of a single chain Fv fragment. A variety of antigen-binding fragments of antibodies can be prepared using antibody genes in which one or more stop codons has been introduced upstream of the natural stop site. For example, an expression construct encoding a F(ab′)₂ portion of an immunoglobulin heavy chain can be designed by introducing a translation stop codon at the 3′ end of the sequence encoding the hinge region of the heavy chain.

The invention also relates to the individual heavy and light chains of the antibodies (e.g., mouse antibodies, human antibodies, humanized antibodies, chimeric antibodies) that bind an αE integrin and to antigen-binding portions thereof. The heavy chains or light chains (and antigen-binding portions thereof) of the invention can bind an αE integrin when paired with a complementary light or heavy chain, respectively. Complementary chains can be identified using any suitable method (e.g., phage display, transgenic animals). For example, a transgenic animal comprising a functionally rearranged nucleic acid encoding a desired heavy chain can be prepared. The heavy-chain transgenic animal can be immunized with the antigen of interest and hybridomas produced. Because of allelic exclusion at immunoglubulin loci, the heavy-chain transgenic mouse may not significantly express endogenous heavy chains and substantially all antibodies elicited by immunization can comprise the heavy chain of interest and a complementary light chain.

The antigen-binding properties (e.g., specificity, affinity) of antibodies and antigen-binding fragments of antibodies can be elucidated using any suitable method. For example, binding specificity can be determined using assays in which formation of a complex between antibody or antigen-binding fragment and an αE integrin, such as an αEβ7 integrin, is detected or measured. Compositions which comprise an αE integrin and which can be used to assess antigen-binding properties of the antibodies and antigen-binding fragments described herein include, a membrane fraction of a cell comprising an αEβ7 integrin, a cell bearing an αEβ7 integrin, such as a human lymphocyte, human lymphocyte cell line or recombinant host cell comprising a nucleic acid encoding αE and/or β7 which expresses an αEβ7 integrin, a recombinant soluble αEβ7, such as ts.αEβ7.coil described herein, and the like. Binding and/or adhesion assays or other suitable methods can also be used in procedures for the identification and/or isolation of antibodies (e.g., human and/or humanized antibodies) having the requisite specificity (e.g., an assay in which adhesion between a cell bearing an αEβ7 integrin and a ligand thereof (e.g., a second cell expressing E-cadherin, an immobilized E-cadherin fusion protein (e.g., E-cadherin-Fc fusion protein) is detected and/or measured), or other suitable methods.

The antibodies of the invention bind an αE integrin (e.g., αEβ7) and preferably bind integrin αE chain (CD103). In a preferred embodiment, the antibody or antigen-binding fragment selectively binds an activation-induced epitope on an integrin αE chain (CD103). The activation-induced epitope can be induced by activation with a divalent cation, such as Mn²⁺, Mg⁺, Ca²⁺ or any combination of the foregoing. The activation-induced epitope on an integrin αE chain expressed on the surface of a cell (e.g., as integrin αEβ7) can also be induced by exposing the cell to phorbol esters (e.g., PMA), or suitable mitogens and/or growth factors. When the cell expressing an integrin αE chain is a T cell, the activation-induced epitope can be induced by signals transduced through the T cell receptor complex. Thus, antibodies that selectively bind an activation-induced epitope can be used to detect or identify activated T cells that express an αE integrin for diagnostic and/or therapeutic purposes.

In one embodiment, the antibody of the invention binds an activation-induced epitope that is induced by exposure of the αE integrin to a divalent cation. Such antibodies bind an integrin αE chain (CD103) in the presence of a divalent cation, such as Mn²⁺, but do not significantly bind an integrin αE chain in the absence of a divalent cation or in the presence of a suitable divalent cation chelating agent (e.g., EDTA).

In certain embodiments, the antibody selectively binds an activation-induced epitope on an integrin αE chain that comprises amino acid residues in the I domain (amino acids 199-390 of SEQ ID NO:2) of integrin αE chain.

In other embodiments, the antibody binds an αE integrin (e.g., selectively binds an activation-induced epitope on integrin αE chain) and inhibits binding of ligand, such as E-cadherin, to the αE integrin (e.g., αEβ7 integrin). For example, the antibody can inhibit αE integrin mediated adhesion of a cell expressing an αE integrin (e.g., αEβ7) to cells expressing a ligand for an αE integrin (e.g. E-cadherin), such as epithelial cells and/or endothelial cells. Preferably, the antibodies do not bind the X domain of integrin αE chain (amino acids 144-198 of SEQ ID NO: 2).

Preferred antibodies that bind an αE integrin (e.g., selectively bind an activation-induced epitope on an integrin αE chain) include chimeric antibodies, humanized antibodies and antigen-binding fragments of the foregoing. Particularly preferred antibodies are human antibodies and antigen-binding fragments of human antibodies.

As described herein, human antibodies designated mAb 3G6, mAb 5E4 and mAb 8D5 which bind integrin αE chain (CD103) have been produced. mAb 3G6 and mAb 5E4 were originally produced as IgM antibodies and mAb 8D5 was originally produced as an IgG2 antibody. As described herein, IgG1 forms of mAbs 3G6, 5E4 and 8D5 have also been produced.

mAb 3G6 (IgM) can be produced by hybridoma 3G6, also referred to as hybridoma 241 3G6.1.15, which was deposited on Apr. 3, 2002, on behalf of Millennium Pharmaceuticals Inc., 75 Sidney Street, Cambridge, Mass., 02139, USA, at the American Type Culture Collection, 10801 University Boulevard, Manassas, Va. 20110, U.S.A., under Accession No. PTA-4201. The invention relates to hybridoma 3G6, to the antibody it produces, antigen-binding fragments thereof, and to nucleic acids encoding the antibody and portions thereof (e.g., heavy chain, heavy chain variable region, light chain, light chain variable region).

An IgG1 form of mAb 3G6 can be produced by “3G6 CHO stable cell line,” also referred to as CHO 3G6 C1.2D6, which was deposited on Apr. 3, 2002, on behalf of Millennium Pharmaceuticals Inc., 75 Sidney Street, Cambridge, Mass., 02139, USA, at the American Type Culture Collection, 10801 University Boulevard, Manassas, Va. 20110, U.S.A., under Accession No. PTA-4204. The invention relates to cell line CHO 3G6 C1.2D6, to the antibody it produces, antigen-binding fragments thereof, and to nucleic acids encoding the antibody and portions thereof (e.g., heavy chain, heavy chain variable region, light chain, light chain variable region).

mAb 5E4 can be produced by hybridoma 5E4, also referred to as hybridoma 233 5E4.3.10, which was deposited on Apr. 3, 2002, on behalf of Millennium Pharmaceuticals Inc., 75 Sidney Street, Cambridge, Mass., 02139, USA, at the American Type Culture Collection, 10801 University Boulevard, Manassas, Va. 20110, U.S.A., under Accession No. PTA-4202. The invention relates to hybridoma 5E4, to the antibody it produces, antigen-binding fragments thereof, and to nucleic acids encoding the antibody and portions thereof (e.g., heavy chain, heavy chain variable region, light chain, light chain variable region).

An IgG1 form of mAb 5E4 can be produced by “5E4 CHO stable cell line,” also referred to as CHO 5G4 A1.2C12, which was deposited on Apr. 3, 2002, on behalf of Millennium Pharmaceuticals Inc., 75 Sidney Street, Cambridge, Mass., 02139, USA, at the American Type Culture Collection, 10801 University Boulevard, Manassas, Va. 20110, U.S.A., under Accession No. PTA-4205. The invention relates to cell line CHO 5G4 A1.2C12, to the antibody it produces, antigen-binding fragments thereof, and to nucleic acids encoding the antibody and portions thereof (e.g., heavy chain, heavy chain variable region, light chain, light chain variable region).

mAb 8D5 can be produced by hybridoma 8D5, also referred to as hybridoma 321 8D5.3.11.8, which was deposited on Apr. 3, 2002, on behalf of Millennium Pharmaceuticals Inc., 75 Sidney Street, Cambridge, Mass., 02139, USA, at the American Type Culture Collection, 10801 University Boulevard, Manassas, Va. 20110, U.S.A., under Accession No. PTA-4203. The invention relates to hybridoma 8D5, to the antibody it produces, antigen-binding fragments thereof, and to nucleic acids encoding the antibody and portions thereof (e.g., heavy chain, heavy chain variable region, light chain, light chain variable region). As described herein, hybridoma 8D5 produces an IgG2 antibody.

The antibodies and antigen-binding fragments of the invention can bind to the same or similar epitope as mAb 3G6, mAb 5E4 or mAb 8D5. Antibodies and antigen-binding fragments that bind the same or similar epitope as mAb 3G6, mAb 5E4 or mAb 8D5 be identified using any suitable method, such as a competitive binding assay. For example, as described herein, an antibody can be tested for the ability to competitively inhibit binding of mAb 3G6, mAb 5E4 or mAb 8D5 to a fusion protein comprising the I domain of integrin αE chain or to an αE integrin (e.g., αEβ7) expressed on the surface of a cell. Competitive inhibition of binding of mAb 3G6, mAb 5E4 or mAb 8D5 in this type of assay is indicative that the test antibody binds the same or similar epitope as mAb 3G6, mAb 5E4 or mAb 8D5.

In particular embodiments, the antibody can have the epitopic specificity of mAb 3G6, mAb 5E4 or mAb 8D5. The fine epitopic specificity of an antibody can be determined using any suitable method, such as mutational analysis. For example, as described herein, a series of integrin αE chain variants comprising amino acid replacements can be prepared and an antibody can be tested for the ability to bind each variant. Inhibited or abrogated binding to a variant comprising a particular amino acid substitution is indicative that the substituted amino acid is part of the epitope that the antibody binds. (See, Higgins et al., J. Biol. Chem. 275:25652-25664 (2000).) In one embodiment, the antibody of the invention has the epitopic specificity of mAb 8D5 and binds an epitope that comprises Phe298 of integrin αE chain (SEQ ID NO: 1).

In more particular embodiments, the antibody comprises one, two or three heavy chain complementarity determining regions (HCDR1, HCDR2 and/or HCDR3) having the amino acid sequences of the heavy chain CDRs of mAb 3G6 wherein, optionally, one or two amino acids in each heavy chain CDR can be conservatively substituted, and one, two or three light chain complementarity determining regions (LCDR1, LCDR2 and/or LCDR3) having the amino acid sequences of the light chain CDRs of mAb 3G6 wherein, optionally, one or two amino acids in each light chain CDR can be conservatively substituted. Preferably, the antibody comprises the three heavy chain CDRs and the three light chain CDRs of mAb 3G6. In more particular embodiments, the antibody comprises the heavy chain variable region of mAb 3G6 (SEQ ID NO: 4) and the light chain variable region of mAb 3G6 (SEQ ID NO: 9).

In other particular embodiments, the antibody comprises one, two or three heavy chain complementarity determining regions (HCDR1, HCDR2 and/or HCDR3) having the amino acid sequences of the heavy chain CDRs of mAb 5E4 wherein, optionally, one or two amino acids in each heavy chain CDR can be conservatively substituted, and one, two or three light chain complementarity determining regions (LCDR1, LCDR2 and/or LCDR3) having the amino acid sequences of the light chain CDRs of mAb 5E4 wherein, optionally, one or two amino acids in each light chain CDR can be conservatively substituted. Preferably, the antibody comprises the three heavy chain CDRs and the three light chain CDRs of mAb 5E4. In more particular embodiments, the antibody comprises the heavy chain variable region of mAb 5E4 (SEQ ID NO: 14) and the light chain variable region of mAb 5E4 (SEQ ID NO: 19).

In additional particular embodiments, the antibody comprises one, two or three heavy chain complementarity determining regions (HCDR1, HCDR2 and/or HCDR3) having the amino acid sequences of the heavy chain CDRs of mAb 8D5 wherein, optionally, one or two amino acids in each heavy chain CDR can be conservatively substituted, and one, two or three light chain complementarity determining regions (LCDR1, LCDR2 and/or LCDR3) having the amino acid sequences of the light chain CDRs of mAb 8D5 wherein, optionally, one or two amino acids in each light chain CDR can be conservatively substituted. Preferably, the antibody comprises the three heavy chain CDRs and the three light chain CDRs of mAb 8D5. In more particular embodiments, the antibody comprises the heavy chain variable region of mAb 8D5 (SEQ ID NO: 24) and the light chain variable region of mAb 8D5 (SEQ ID NO: 29).

In additional embodiments, the invention provides novel heavy chains and light chains of the antibodies and antigen-binding fragments described herein. In particular embodiments, the antibody heavy chains or antigen-binding portions thereof comprise at least two and preferably three CDRs having the amino acid sequences of the heavy chain CDRs of mAb 3G6, the heavy chain CDRs of mAb 5E4 or the heavy chain CDRs of mAb 8D5. Optionally, one or two amino acid residues in each heavy chain CDR can be conservatively substituted. In preferred embodiments, the antibody heavy chains or antigen-binding portions thereof comprise three CDRs that have the amino acid sequences of the three CDRs of the heavy chain of mAb 3G6, the three CDRs of the heavy chain of mAb 5E4 or the three CDRs of the heavy chain of mAb 8D5. In other embodiments, the antibody heavy chains or antigen-binding portions thereof comprise the heavy chain variable region of mAb 3G6, mAb 5E4 or mAb 8D5. For example, the antibody heavy chains can comprise an amino acid sequence selected from the group consisting of SEQ ID NO: 4, SEQ ID NO: 14 and SEQ ID NO: 24. The antibody heavy chains and portions thereof can comprise any suitable framework regions and/or constant regions (as described herein).

In certain embodiments, the antibody light chains or antigen-binding portions thereof comprise at least two and preferably three CDRs having the amino acid sequences of the light chain CDRs of mAb 3G6, or the light chain CDRs of mAb 5E4 or the light chain CDRs of mAb 8D5. Optionally, one or two amino acid residues in each light chain CDR can be conservatively substituted. In preferred embodiments, the antibody light chains or antigen-binding portions thereof comprise three CDRs that have the amino acid sequences of the three CDRs of the light chain of mAb 3G6, the three CDRs of the light chain of mAb 5E4 or the three CDRs of the light chain of mAb 8D5. In other embodiments, the antibody light chains or antigen-binding portions thereof comprise the light chain variable region of mAb 3G6, mAb 5E4 or mAb 8D5. For example, the antibody light chains can comprise an amino acid sequence selected from the group consisting of SEQ ID NO: 9, SEQ ID NO: 19 and SEQ ID NO: 29. The antibody light chains and portions thereof can comprise any suitable framework regions and/or constant regions (as described herein).

Fusion Proteins and Immuno-Conjugates

Fusion proteins and immunoconjugates can be produced in which an antibody moiety (e.g., antibody or antigen-binding fragment thereof, antibody chain or antigen-binding portion thereof) is linked directly or indirectly to a non-immunoglobulin moiety (i.e., a moiety which does not occur in immunoglobulins as found in nature). Fusion proteins comprise an antibody moiety and a non-immunoglobulin moiety that are components of a single continuous polypeptide chain. The non-immunoglobulin moiety can be located N-terminally, C-terminally or internally with respect to the antibody moiety. For example, some embodiments can be produced by the insertion of a nucleic acid encoding immunoglobulin sequences into a suitable expression vector, such as a pET vector (e.g., pET-15b, Novagen), a phage vector (e.g., pCANTAB 5 E, Pharmacia), or other vector (e.g., pRIT2T Protein A fusion vector, Pharmacia). The resulting construct can be expressed (e.g., in vivo by a suitable host cell, in vitro) to produce antibody chains that comprise a non-immunoglobulin moiety (e.g., Histidine tag, E tag, Protein A IgG binding domain). Fusion proteins can be isolated or recovered using any suitable technique, such as chromatography using a suitable affinity matrix (see e.g., Current Protocols in Molecular Biology (Ausubel, F. M. et al., eds., Vol. 2, Suppl. 26, pp. 16.4.1-16.7.8 (1991)).

In other embodiments, the antibody moiety and non-immunoglobulin moiety may not be part of a continuous polypeptide chain, but can be connected or conjugated directly or indirectly through any suitable linker. Suitable methods for connecting or conjugating the moieties are well known in the art. (See, e.g., Ghetie et al., Pharmacol. Ther. 63:209-34 (1994)). A variety of suitable linkers (e.g., heterobifunctional reagents) and methods for preparing immuno-conjugates are well known in the art. (See, for example, Hermanson, G. T., Bioconjugate Techniques, Academic Press: San Diego, Calif. (1996).) Suitable non-immunoglobulin moieties for inclusion in an immuno-conjugate include a therapeutic moiety such as a toxin (e.g., cytotoxin, cytotoxic agent), a therapeutic agent (e.g., a chemotherapeutic agent, an antimetabolite, an alkylating agent, an anthracycline, an antibiotic, an anti-mitotic agent, a biological response modifier (e.g., a cytokine (e.g., an interleukin, an interferon, a tumor necrosis factor), a growth factor (e.g., a neurotrophic factor)), a plasminogen activator), a radionuclide (e.g, a radioactive ion), an enzyme and the like. Suitable cytotoxins or cytotoxic agents include any agent that is detrimental to cells. Examples of suitable cytotoxins or cytotoxic agents include TAXOL (paclitaxel, Bristol-Myers Squibb Company), cytochalasin B, gramicidin D, ethidium bromide, emetine, mitomycin (e.g, mitomycin C), etoposide, tenoposide, vincristine, vinblastine, colchicine, doxorubicin, daunorubicin, dihydroxy anthracindione, mitoxantrone, mithramycin, actinomycin D, 1-dehydrotestosterone, glucocorticoids, procaine, tetracaine, lidocaine, propranolol, puromycin, maytansinoids (e.g., maytansinol (see U.S. Pat. No. 5,208,020), CC-1065 (see U.S. Pat. Nos. 5,475,092, 5,585,499, 5,846,545), DM1) and analogs or homologs of any of the forgoing agents. Suitable therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepachlorambucil, CC-1065, melphalan, carmustine (BSNU), lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, TAXOL (paclitaxel, Bristol-Myers Squibb Company) and maytansinoids (e.g., maytansinol (see U.S. Pat. No. 5,208,020), CC-1065 (see U.S. Pat. Nos. 5,475,092, 5,585,499, 5,846,545), DM1)). Suitable radionuclides include, for example iodine (e.g., iodine-125, -126) yttrium (e.g., yttrium-90, -91) and praseodymium (e.g., praseodymium-144, -145).

In certain embodiments, the therapeutic agent can be a protein or polypeptide possessing a desired biological activity. Such proteins or polypeptides can include, for example, a toxin such as abrin, ricin A, pseudomonas exotoxin, or diphtheria toxin; a protein such as a tumor necrosis factor (e.g., TNFα, TNFβ), and interferon (e.g., α-interferon, β-interferon, γ-interferion), a neurotrophic factor (e.g., nerve growth factor), a growth factor (e.g., platelet derived growth factor), a plasminogen activator (e.g., tissue plasminogen activator); or biological response modifiers such as, for example, cytokines and lymphokines, (e.g., interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”)), or other growth factors. In other embodiments, the antibody or antigen-binding fragment of the invention can be conjugated to a second antibody or antigen-binding fragment to form an antibody heteroconjugate. (See, e.g., U.S. Pat. No. 4,676,980 (Segal).)

Nucleic Acids and Constructs

The present invention also relates to isolated and/or recombinant (including, e.g., essentially pure) nucleic acids comprising sequences which encode an antibody or antigen-binding fragment (e.g., a human, humanized, chimeric antibody or light or heavy chain of any of the foregoing) or fusion protein of the invention.

Nucleic acids referred to herein as “isolated” are nucleic acids which have been separated away from other material (e.g., other nucleic acids such as genomic DNA, cDNA and/or RNA) in its original environment (e.g., in cells or in a mixture of nucleic acids such as a library). An isolated nucleic acid can be isolated as part of a vector (e.g., a plasmid). Nucleic acids can be naturally occurring, produced by chemical synthesis, by combinations of biological and chemical methods (e.g., semisynthetic), and be isolated using any suitable methods.

Nucleic acids referred to herein as “recombinant” are nucleic acids which have been produced by recombinant DNA methodology, including methods which rely upon artificial recombination, such as cloning into a vector or chromosome using, for example, restriction enzymes, homologous recombination, viruses and the like, and nucleic acids prepared using the polymerase chain reaction (PCR). “Recombinant” nucleic acids are also those that result from recombination of endogenous or exogenous nucleic acids through the natural mechanisms of cells or cells modified to allow recombination (e.g., cells modified to express Cre or other suitable recombinase), but are selected for after the introduction to the cells of nucleic acids designed to allow and make recombination probable. For example, a functionally rearranged human-antibody transgene is a recombinant nucleic acid.

The present invention also relates more specifically to nucleic acids that encode the heavy chains and/or light chains of the antibodies and antigen-binding portions described herein. For example, in one embodiment, the nucleic acid can encode a heavy chain or antigen-binding portion thereof that comprises at least one, two or preferably three CDRs having the amino acid sequences of the heavy chain CDRs of mAb 3G6 wherein, optionally, one or two amino acids in each CDR can be conservatively substituted. In another embodiment, the nucleic acid can encode a heavy chain or antigen-binding portion thereof that comprises at least one, two or preferably three CDRs having the amino acid sequences of the heavy chain CDRs of mAb 5E4 wherein, optionally, one or two amino acids in each CDR can be conservatively substituted. In another embodiment, the nucleic acid can encode a heavy chain or antigen-binding portion thereof that comprises at least one, two or preferably three CDRs having the amino acid sequences of the heavy chain CDRs of mAb 8D5 wherein, optionally, one or two amino acids in each CDR can be conservatively substituted. In preferred embodiments, the nucleic acid encodes an antibody heavy chain or antigen-binding portion thereof that comprises three CDRs that have the amino acid sequences of the three CDRs of the heavy chain of mAb 3G6, the three CDRs of the heavy chain of mAb 5E4 or the three CDRs of the heavy chain of mAb 8D5. In other embodiments, the nucleic acid encodes an antibody heavy chain or antigen-binding portion thereof that comprises the heavy chain variable region of mAb 3G6, mAb 5E4 or mAb 8D5. For example, the nucleic acid can comprise a nucleotide sequence selected from the group consisting of SEQ ID NO: 3, SEQ ID NO: 13 and SEQ ID NO: 23. The antibody heavy chains and portions thereof can comprise any suitable framework regions and/or constant regions (as described herein).

In another embodiment, the nucleic acid can encode a light chain or antigen-binding portion thereof that comprises at least one, two or preferably three CDRs having the amino acid sequences of the light chain CDRs of mAb 3G6 wherein, optionally, one or two amino acids in each CDR can be conservatively substituted. In another embodiment, the nucleic acid can encode a light chain or antigen-binding portion thereof that comprises at least one, two or preferably three CDRs having the amino acid sequences of the light chain CDRs of mAb 5E4 wherein, optionally, one or two amino acids in each CDR can be conservatively substituted. In another embodiment, the nucleic acid can encode a light chain or antigen-binding portion thereof that comprises at least one, two or preferably three CDRs having the amino acid sequences of the light chain CDRs of mAb 8D5 wherein, optionally, one or two amino acids in each CDR can be conservatively substituted. In preferred embodiments, the nucleic acid encodes an antibody light chain or antigen-binding portion thereof that comprises three CDRs that have the amino acid sequences of the three CDRs of the light chain of mAb 3G6, the three CDRs of the light chain of mAb 5E4 or the three CDRs of the light chain of mAb 8D5. In other embodiments, the nucleic acid encodes an antibody light chain or antigen-binding portion thereof that comprises the light chain variable region of mAb 3G6, mAb 5E4 or mAb 8D5. For example, the nucleic acid can comprise a nucleotide sequence selected from the group consisting of SEQ ID NO: 8, SEQ ID NO: 18 and SEQ ID NO: 28. The antibody light chains and portions thereof can comprise any suitable framework regions and/or constant regions (as described herein).

Nucleic acid molecules of the present invention can be used in the production of antibodies (e.g., human antibodies, humanized antibodies, chimeric antibodies and antigen-binding fragments of the foregoing) that bind an αE integrin or integrin αE chain (CD103). For example, a nucleic acid (e.g., DNA) encoding an antibody of the invention can be incorporated into a suitable construct (e.g., an expression vector) for further manipulation or for production of the encoded polypeptide in suitable host cells.

Expression constructs or expression vectors suitable for the expression of a antibody or antigen-binding fragment that binds an αE integrin are also provided. For example, a nucleic acid encoding all or part of a desired antibody can be inserted into a nucleic acid vector, such as a plasmid or virus, for expression. The vector can be capable of replication in a suitable biological system (e.g., a replicon). A variety of suitable vectors are known in the art, including vectors which are maintained in single copy or multiple copy, or which become integrated into the host cell chromosome.

Suitable expression vectors can contain a number of components, for example, an origin of replication, a selectable marker gene, one or more expression control elements, such as a transcription control element (e.g., promoter, enhancer, terminator) and/or one or more translation signals, a signal sequence or leader sequence, and the like. Expression control elements and a signal or leader sequence, if present, can be provided by the vector or other source. For example, the transcriptional and/or translational control sequences of a cloned nucleic acid encoding an antibody chain can be used to direct expression.

A promoter can be provided for expression in a desired host cell. Promoters can be constitutive or inducible. For example, a promoter can be operably linked to a nucleic acid encoding an antibody, antibody chain or portion thereof, such that it directs transcription of the nucleic acid. A variety of suitable promoters for procaryotic (e.g., lac, tac, T3, T7 promoters for E. coli) and eucaryotic (e.g., simian virus 40 early or late promoter, Rous sarcoma virus long terminal repeat promoter, cytomegalovirus promoter, adenovirus late promoter, EG-1a promoter) hosts are available.

In addition, expression vectors typically comprise a selectable marker for selection of host cells carrying the vector, and, in the case of a replicable expression vector, an origin or replication. Genes encoding products which confer antibiotic or drug resistance are common selectable markers and may be used in procaryotic (e.g., β-lactamase gene (ampicillin resistance), Tet gene for tetracycline resistance) and eucaryotic cells (e.g., neomycin (G418 or geneticin), gpt (mycophenolic acid), ampicillin, or hygromycin resistance genes). Dihydrofolate reductase marker genes permit selection with methotrexate in a variety of hosts. Genes encoding the gene product of auxotrophic markers of the host (e.g., LEU2, URA3, HIS3) are often used as selectable markers in yeast. Use of viral (e.g., baculovirus) or phage vectors, and vectors which are capable of integrating into the genome of the host cell, such as retroviral vectors, are also contemplated.

Suitable expression vectors for expression in mammalian cells include, for example, pCDM8, pCDNA1.1/amp, pcDNA3.1, pRc/RSV, pEF-1 (Invitrogen, Carlsbad, Calif.), pCMV-SCRIPT, pFB, pSG5, pXT1 (Stratagene, La Jolla, Calif.), pCDEF3 (Goldman, L. A., et al., Biotechniques, 21:1013-1015 (1996)), pSVSPORT (GibcoBRL, Rockville, Md.), pEF-Bos (Mizushima, S., et al., Nucleic Acids Res., 18:5322 (1990)) and the like. Expression vectors which are suitable for use in various expression hosts, such as prokaryotic cells (E. coli), insect cells (Drosophila Schnieder S2 cells, Sf9) and yeast (P. methanolica, P. pastoris, S. cerevisiae) are also available.

Thus, the invention provides an expression vector comprising a nucleic acid encoding an antibody, antigen-binding fragment of an antibody (e.g., a human, humanized, chimeric antibody or antigen-binding fragment of any of the foregoing), antibody chain (e.g., heavy chain, light chain) or antigen-binding portion of an antibody chain that binds an αE integrin (e.g., an integrin αE chain (CD103)).

Recombinant Host Cells and Methods of Production

In another aspect, the invention relates to recombinant host cells and a method of preparing an antibody or antigen-binding fragment, antibody chain (e.g., heavy chain, light chain) or antigen-binding portion of an antibody chain, or fusion protein of the invention. The antibody or antigen-binding fragment can be obtained, for example, by the expression of one or more recombinant nucleic acids encoding an antibody, antigen-binding fragment of an antibody, antibody chain or antigen-binding portion of an antibody chain that binds an αE integrin in a suitable host cell, or using other suitable methods. For example, the expression constructs described herein can be introduced into a suitable host cell, and the resulting cell can be maintained (e.g., in culture, in an animal, in a plant) under conditions suitable for expression of the constructs. Suitable host cells can be prokaryotic, including bacterial cells such as E. coli, B. subtilis and/or other suitable bacteria; eucaryotic cells, such as fungal or yeast cells (e.g., Pichia pastoris, Aspergillus sp., Saccharomyces cerevisiae, Schizosaccharomyces pombe, Neurospora crassa), or other lower eukaryotic cells, and cells of higher eucaryotes such as those from insects (e.g., Drosophila Schnieder S2 cells, Sf9 insect cells (WO 94/26087 (O'Connor)), mammals (e.g., COS cells, such as COS-1 (ATCC Accession No. CRL-1650) and COS-7 (ATCC Accession No. CRL-1651), CHO (e.g., ATCC Accession No. CRL-9096), 293 (ATCC Accession No. CRL-1573), HeLa (ATCC Accession No. CCL-2), CV1 (ATCC Accession No. CCL-70), WOP (Dailey, L., et al., J. Virol., 54:739-749 (1985), 3T3, 293T (Pear, W. S., et al., Proc. Natl. Acad. Sci. U.S.A., 90:8392-8396 (1993)) NSO cells, SP2/0, HuT 78 cells and the like, or plants (e.g., tobacco). (See, for example, Ausubel, F. M. et al., eds. Current Protocols in Molecular Biology, Greene Publishing Associates and John Wiley & Sons Inc. (1993).)

The invention also relates to a recombinant host cell which comprises a (one or more) recombinant nucleic acid or expression construct comprising a nucleic acid encoding an antibody, antigen-binding fragment of an antibody (e.g., a human, humanized, chimeric antibody or antigen-binding fragment of any of the foregoing), antibody chain (e.g., heavy chain, light chain) or antigen-binding portion of an antibody chain that binds an αE integrin (e.g., an integrin αE chain (CD103)). In particular embodiments, the recombinant host cell is hybridoma 3G6, hybridoma 5E4, hybridoma 8D5, CHO 3G6 C1.2D6 or CHO 5G4 A1.2C12.

The invention also includes a method of preparing an antibody, antigen-binding fragment of an antibody (e.g., a human, humanized, chimeric antibody or antigen-binding fragment of any of the foregoing), antibody chain (e.g., heavy chain, light chain) or antigen-binding portion of an antibody chain that binds an αE integrin (e.g., an integrin αE chain (CD103)), comprising maintaining a recombinant host cell of the invention under conditions appropriate for expression of an antibody, antigen-binding fragment of an antibody, antibody chain or antigen-binding fragment of an antibody chain. The method can further comprise the step of isolating or recovering the antibody, antigen-binding fragment of an antibody, antibody chain or antigen-binding fragment of an antibody chain, if desired.

For example, a nucleic acid molecule (i.e., one or more nucleic acid molecules) encoding the heavy and light chains of a human antibody that binds an integrin αE chain, or an expression construct (i.e., one or more constructs) comprising such nucleic acid molecule(s), can be introduced into a suitable host cell to create a recombinant host cell using any method appropriate to the host cell selected (e.g., transformation, transfection, electroporation, infection), such that the nucleic acid molecule(s) are operably linked to one or more expression control elements (e.g., in a vector, in a construct created by processes in the cell, integrated into the host cell genome). The resulting recombinant host cell can be maintained under conditions suitable for expression (e.g., in the presence of an inducer, in a suitable animal, in suitable culture media supplemented with appropriate salts, growth factors, antibiotics, nutritional supplements, etc.), whereby the encoded polypeptide(s) are produced. If desired, the encoded protein can be isolated or recovered (e.g., from the animal, the host cell, medium, milk). This process encompasses expression in a host cell of a transgenic animal (see, e.g., WO 92/03918, GenPharm International).

The antibodies, antigen-binding fragments, antibody chains and antigen-binding portions thereof described herein can also be produced in a suitable in vitro expression system, by chemical synthesis or by any other suitable method.

Diagnostic and Therapeutic Methods

The antibodies (including fragments), fusion proteins and immuno-conjugates described herein can bind an αE integrin and can be used to detect, measure, select, isolate and/or purify an αE integrin (e.g., αEβ7 integrin) or variants thereof (e.g., by affinity purification or other suitable methods), and to study αE integrin structure (e.g., conformation) and function. The antibodies, fusion proteins and immuno-conjugates of the present invention can also be used in diagnostic applications (e.g., in vitro, ex vivo) and/or in therapeutic applications.

The antibodies, fusion proteins and immuno-conjugates can be used to detect and/or measure the level of an αE integrin (e.g., αEβ7 integrin) in a sample (e.g., tissues or body fluids, such as an inflammatory exudate, bronchial lavage, blood, serum, bowel fluid, biopsy). In one example, a sample (e.g., tissue and/or body fluid) can be obtained from an individual and a suitable immunological method can be used to detect and/or measure αE integrin expression. Suitable immunological methods for detecting or measuring αE integrin expression include enzyme-linked immunosorbent assays (ELISA), radioimmunoassay, immunohistology, flow cytometry, and the like.

In one embodiment, the invention is a method of detecting or measuring an activated αE integrin in a sample (e.g., a biological sample) comprising contacting a sample (e.g., a biological sample) with an antibody or antigen-binding fragment thereof that binds an activation-induced epitope on an αE integrin (e.g., on an integrin αE chain (CD103)) under conditions suitable for binding of the antibody or antigen-binding fragment to the αE integrin and detecting and/or measuring binding of the antibody or antigen-binding fragment to the αE integrin. Binding of the antibody or antigen-binding fragment thereof to the αE integrin indicates the presence of the αE integrin in the sample. In an application of the method, an antibody or antigen-binding fragment of the invention can be used to analyze normal versus inflamed tissues (e.g., from a human) for activated αE integrin reactivity and/or expression to detect associations between disease (e.g., inflammatory bowel disease, graft rejection) and increased expression of activated αE (e.g., in affected tissues). In embodiments where the antibody or antigen-binding fragment binds an activation-induced epitope, the antibodies, antigen-binding fragments, fusion proteins and immuno-conjugates of the invention can be used to detect, measure, select, isolate and/or purify activated αE integrin or cells expressing an activated αE integrin.

The antibodies, fusion proteins and/or immuno-conjugates of the present invention permit assessment of the presence of an αE integrin in normal versus inflamed tissues, through which the presence or severity of disease, disease progress and/or the efficacy of therapy can be assessed. For example, therapy can be monitored and efficacy assessed. In one example, an αE integrin can be detected and/or measured in a first sample obtained from a subject having an inflammatory disease and therapy can be initiated. Later, a second sample can be obtained from the subject and αE integrin in the sample can be detected and/or measured. A decrease in the quantity of αE integrin detected or measured in the second sample can be indicative of therapeutic efficacy.

The antibodies, fusion proteins and immuno-conjugates described herein can modulate an activity or function of an αE integrin (e.g., αEβ7 integrin), such as ligand binding (e.g., E-cadherin) and/or leukocyte infiltration function, including recruitment and/or accumulation of leukocytes (e.g., T cells) in tissues. Antibodies, fusion proteins and immuno-conjugates that bind an activation-induced epitope can be used to selectively target cells expressing activated αE integrin (e.g., αEβ7 integrin) for therapy. For example, an antibody that binds an activation-induced epitope on an αEβ7 integrin and is capable of activating complement (e.g., a human IgG1 antibody) can be administered to selectively deplete cells expressing activated αEβ7 through, for example, complement-mediated lysis.

Preferably the antibodies, fusion proteins and immuno-conjugates can selectively bind an αE integrin (e.g., αEβ7 integrin) and inhibit αE integrin-mediated interactions, such as αE integrin-mediated adhesion of a cell (e.g., T cell) to endothelial cells. In particularly preferred embodiments, the antibodies, fusion proteins and immuno-conjugates can inhibit the interaction of αEβ7 with E-cadherin.

The antibodies, fusion proteins and immuno-conjugates described herein can be administered to a subject to modulate an inflammatory response or to treat an inflammatory disease or disorder. For example, an antibody which inhibits the binding of an αE integrin to a ligand (i.e., one or more ligands) can be administered in the treatment of diseases associated with leukocyte (e.g., lymphocyte, monocyte) infiltration of tissues, particularly of mucosal tissues. An effective amount of an antibody, fusion protein and/or immuno-conjugate (i.e., one or more) can be administered to a subject (e.g., a mammal, such as a human or other primate) in order to treat such a disease. For example, inflammatory diseases, including diseases which are associated with leukocyte infiltration of the gastrointestinal tract (including gut-associated endothelium), other mucosal tissues, or tissues expressing the molecule E-cadherin (e.g., mucosal epithelial surfaces), can be treated according to the present method. Similarly, an individual having a disease associated with leukocyte infiltration of tissues as a result of binding of leukocytes to cells (e.g., epithelial cells) expressing E-cadherin can be treated according to the present invention.

Examples of inflammatory diseases associated with mucosal tissues which can be treated according to the present method include mastitis (mammary gland), cholecystitis, cholangitis or pericholangitis (bile duct and surrounding tissue of the liver), chronic bronchitis, chronic sinusitis, asthma, and graft versus host disease (e.g., in the gastrointestinal tract). As seen in Crohn's disease, mucosal inflammation often extends beyond the mucosal surface. Accordingly chronic inflammatory diseases of the lung which result in interstitial fibrosis, such as hypersensitivity pneumonitis, collagen diseases, sarcoidosis, and other idiopathic conditions can be amenable to treatment.

According to the method, the severity of symptoms associated with an inflammatory condition can be inhibited (reduced) in whole or in part. When the subject has a relapsing or chronic condition, an effective amount of an antibody, fusion protein and/or immuno-conjugate of the invention can be administered to treat the subject, and therapy can be continued (maintenance therapy) with the same or different dosing as indicated, to inhibit relapse or renewed onset of symptoms. Preferably, the antibodies, fusion proteins and/or immuno-conjugates are administered to treat a subject having a mucosal inflammatory diseases, such as an inflammatory disease of the respiratory tract (e.g., bronchus, lung), urogenital tract (e.g., kidney, urinary bladder) or alimentary canal and associated organs and tissues (e.g., mouth, salivary glands, esophagus, stomach, small intestine, colon, pancreas, liver, gall bladder).

In a particularly preferred embodiment, the subject to be treated has an inflammatory bowel disease (IBD), such as ulcerative colitis, Crohn's disease, ileitis, Celiac disease, nontropical Sprue, enteropathy associated with seronegative arthropathies, colitis (e.g., microscopic or collagenous colitis), gastroenteritis (e.g., eosinophilic gastroenteritis), or pouchitis resulting after proctocolectomy and ileoanal anastomosis. Subjects having pancreatitis or insulin-dependent diabetes mellitus can also be treated using the present method. In another embodiment, the subject to be treated has an has on oral inflammatory disease, Sjogren's syndrome or Behcet's syndrome.

In another embodiment, the subject to be treated has a pulmonary inflammatory disease, such as a chronic obstructive lung disease (e.g., chronic bronchitis, asthma, silicosis, chronic obstructive pulmonary disease), hypersensitivity pneumonitis, pulmonary fibrosis (e.g., idiopathic pulmonary fibrosis) or sarcoidosis. In another embodiment, the subject to be treated has a cutaneous inflammatory disease, such as psoriasis or inflammatory dermatoses.

In another embodiment, the invention is a method of inhibiting graft rejection (e.g., allograft rejection, xenograft rejection) or graft versus host disease, comprising administering to a subject in need thereof an effective amount of an antibody, fusion protein and/or immuno-conjugate of the invention. In particular embodiments, the transplanted graft is a mucosa-associated organ or tissue, such as kidney, liver, lung and the like.

The invention also relates to a method of inhibiting αE integrin (e.g. αEβ7 integrin) mediated homing of leukocytes in a subject, comprising to a subject in need thereof an effective amount of an antibody, fusion protein and/or immuno-conjugate of the invention. For example, the homing of leukocytes to mucosal sites (e.g., gut, lung) can be inhibited.

As used herein, “subject” refers to humans and animals such as mammals, including, primates, cows, sheep, goats, horses, dogs, cats, rabbits, guinea pigs, rats, mice or other bovine, ovine, equine, canine, feline, rodent or murine species.

Diseases and conditions associated with inflammation, infection, and cancer can be treated using the method. In a preferred embodiment, the disease or condition is one in which the actions of cells bearing an αE integrin (e.g., αEβ7), such as lymphocytes (e.g., activated or stimulated T lymphocytes), are to be inhibited or promoted for therapeutic or prophylactic purposes.

Diseases or conditions, including chronic diseases, of humans or other species which can be treated with the antibodies, fusion proteins and/or immuno-conjugates of the invention, include, but are not limited to:

inflammatory or allergic diseases and conditions, including systemic anaphylaxis or hypersensitivity responses, drug allergies (e.g., to penicillin, cephalosporins), insect sting allergies; inflammatory bowel diseases, such as Crohn's disease, ulcerative colitis, celiac disease, ileitis and enteritis; sarcoidosis; vaginitis; psoriasis and inflammatory dermatoses such as dermatitis, eczema, atopic dermatitis, allergic contact dermatitis, urticaria; vasculitis (e.g., necrotizing, cutaneous, and hypersensitivity vasculitis); spondyloarthropathies; scleroderma; respiratory allergic diseases such as asthma, allergic rhinitis, hypersensitivity lung diseases, hypersensitivity pneumonitis, interstitial lung diseases (ILD) (e.g., idiopathic pulmonary fibrosis, or ILD associated with rheumatoid arthritis, or other autoimmune conditions);

autoimmune diseases, such as arthritis (e.g., rheumatoid arthritis, psoriatic arthritis), multiple sclerosis, systemic lupus erythematosus, myasthenia gravis, diabetes, including diabetes mellitus and juvenile onset diabetes, glomerulonephritis and other nephritides, autoimmune thyroiditis, Behcet's syndrome;

graft rejection (e.g., in transplantation), including allograft rejection or graft-versus-host disease;

viral infection, e.g., infection by hepatitis C virus (HCV), human papilloma virus (HPV), respiratory syncytial virus, influenza virus, simian immunodeficiency virus (SIV) or human immunodeficiency virus (HIV);

cancers and/or neoplastic diseases, such as leukemias and lymphomas;

other diseases or conditions in which undesirable inflammatory responses are to be inhibited can be treated, including, but not limited to, atherosclerosis (e.g., transplant accelerated atherosclerosis), restenosis, cytokine-induced toxicity, myositis (including polymyositis, dermatomyositis).

Modes of Administration

According to the method, an (i.e., one or more) antibody, antigen-binding fragment thereof, fusion protein and/or immuno-conjugate can be administered to the subject by an appropriate route, either alone or in combination with another drug. An “effective amount” of antibody, fusion protein and/or immuno-conjugate is administered. An “effective amount” is an amount sufficient to achieve the desired therapeutic or prophylactic effect, under the conditions of administration, such as an amount sufficient to inhibit binding of αE integrin (αEβ7 integrin) to E-cadherin expressed on epithelial cells, and thereby, inhibit αE integrin-mediated function, such as leukocyte binding, extravasation and/or retention (e.g., as intra-epithelial lymphocytes (IEL)). The antibody, fusion protein and/or immuno-conjugate can be administered in a single dose or multiple doses. The antibody or antigen-binding fragment can be administered as a bolus and/or infusion (e.g., continuous infusion). The dosage can be determined by methods known in the art and is dependent, for example, upon the antibody, antigen-binding fragment, fusion protein and/or immuno-conjugate chosen, the subject's age, sensitivity and tolerance to drugs, and overall well-being. Typically, an effective amount can range from about 0.01 mg per day to about 100 mg per day for an adult. Preferably, the dosage ranges from about 1 mg per day to about 100 mg per day or from about 1 mg per day to about 10 mg per day. Human, humanized and chimeric antibodies can often be administered with less frequency than other types of therapeutics. For example, an effective amount of a human, humanized or chimeric antibody (or antigen-binding fragment of any of the foregoing) can range from about 0.01 mg/kg to about 5 or 10 mg/kg administered daily, weekly, biweekly or monthly.

A variety of routes of administration are possible including, for example, oral, dietary, topical, transdermal, rectal, parenteral (e.g., intravenous, intraarterial, intramuscular, subcutaneous, intradermal, intraperatoneal injection), and inhalation (e.g., intrabronchial, intranasal or oral inhalation, intranasal drops) routes of administration, depending on the agent and disease or condition to be treated. Administration can be local or systemic as indicated. The preferred mode of administration can vary depending upon the agent chosen, and the condition (e.g., disease) being treated, however, oral or parenteral administration is generally preferred.

The antibody, fusion protein and/or immuno-conjugate and any other therapeutic agent to be administered can be administered as a neutral compound or as a salt. Salts of compounds (e.g., an antibody) containing an amine or other basic group can be obtained, for example, by reacting with a suitable organic or inorganic acid, such as hydrogen chloride, hydrogen bromide, acetic acid, perchloric acid and the like. Compounds with a quaternary ammonium group also contain a counteranion such as chloride, bromide, iodide, acetate, perchlorate and the like. Salts of compounds containing a carboxylic acid or other acidic functional group can be prepared by reacting with a suitable base, for example, a hydroxide base. Salts of acidic functional groups contain a countercation such as sodium, potassium and the like.

The antibody, fusion protein and/or immuno-conjugate can be administered to the individual as part of a pharmaceutical composition for modulation (e.g., inhibition) of αE integrin function (e.g., ligand binding and/or leukocyte infiltration), or treating a subject having a disease described herein. The pharmaceutical composition can comprise an antibody, antigen-binding fragment, fusion protein and/or immuno-conjugate of the invention and a pharmaceutically or physiologically acceptable carrier. Formulation will vary according to the route of administration selected (e.g., solution, emulsion, capsule). Suitable pharmaceutical and physiological carriers can contain inert ingredients which do not interact with the antibody, fusion protein and/or immuno-conjugate. Standard pharmaceutical formulation techniques can be employed, such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% benzyl alcohol), phosphate-buffered saline, Hank's solution, Ringer's-lactate and the like. Methods for encapsulating compositions (such as in a coating of hard gelatin or cyclodextran) are known in the art (Baker, et al, “Controlled Release of Biological Active Agents”, John Wiley and Sons, 1986). For inhalation, the agent can be solubilized and loaded into a suitable dispenser for administration (e.g., an atomizer, nebulizer or pressurized aerosol dispenser).

Furthermore, the antibody or fusion protein of the invention and other therapeutic agents that are proteins can be administered via in vivo expression of the recombinant protein. In vivo expression can be accomplished via somatic cell expression according to suitable methods (see, e.g. U.S. Pat. No. 5,399,346). In this embodiment, a nucleic acid encoding the protein can be incorporated into a retroviral, adenoviral or other suitable vector (preferably, a replication deficient infectious vector) for delivery, or can be introduced into a transfected or transformed host cell capable of expressing the protein for delivery. In the latter embodiment, the cells can be implanted (alone or in a barrier device), injected or otherwise introduced in an amount effective to express the protein in a therapeutically effective amount.

The present invention will now be illustrated by the following Examples, which are not intended to be limiting in any way.

### EXAMPLES

### Methods and Materials

E-cadherin-IgG Fusion Protein

A DNA fragment encoding human E-cadherin extracellular domain (residues 1-695 of SEQ ID NO: 34) was isolated by PCR using full-length E-cadherin cDNA as template. Synthetic primers Spe-ECAD(5) (gcactagtccaccatgggcccttggagccgc; SEQ ID NO: 42) and ECAD-XHO(3) (ccctcgagaggctgtgccttcctaca; SEQ ID NO: 43) were designed so that SpeI and XhoI restriction sites were incorporated at the 5′ and 3′ of the PCR product, respectively. The PCR product was digested with SpeI and XhoI.

A DNA fragment coding for an human IgG Fc fragment (including the hinge, CH2 and CH3) was isolated by PCR using a fusion construct that encodes a fusion protein that contains a human IgG1 constant region that has been mutated to inhibit binding to Fc receptor as template and synthetic primers Xho-IgG(5) (atctcgagcccaaatcttgtgac; SEQ ID NO: 44) and IgGNot(3) (tagcggccgctcatttacccggagacag; SEQ ID NO: 45) which introduced XhoI and NotI sites at the 5′ and 3′ ends of the product, respectively. The product was cut with XhoI and NotI.

The PCR products (E-cadherin and IgG Fc) were ligated into vector pCDEF3 (Goldman, L. A., et al., Biotechniques, 21:1013-1015 (1996)) that had been linearized with SpeI and NotI. Vector pCDEF3 is a derivative of pcDNA (Invitrogen, Carlsbad, Calif.) and contains the EF-1 promoter. The sequence of the resulting E-cadherin-IgG fusion construct in pCDEF3 was confirmed by DNA sequencing.

The fusion construct encoded a fusion protein that contained a Leucine residue between the E-cadherin portion and the IgG1 Fc portion, and the IgG1 Fc portion contained mutations to reduce binding to Fc receptor.

Expression and Purification

The E-cadherin-IgG fusion construct was transiently transfected in 293T cells using calcium phosphate transfection method. 10 μg of the expression vector was used to transfect one 10 cm plate of 293T cells (Pear, W. S., et al., Proc. Natl. Acad. Sci. U.S.A., 90:8392-8396 (1993)). For large scale purification of the fusion protein, 30-35 plates of cells were typically transfected. 7-11 hours post-transfection, the culture medium was changed to media supplemented with 10% ultra low IgG fetal bovine serum (Gibco). The transfected cells were cultured and the culture supernatant (10 mL) was collected daily for three days. The human IgG was isolated from the collected supernatant by chromatography using a protein A column at 4° C. The column was washed with TBS/Ca (20 mM Tris, pH 7.5, 140 mM NaCl, supplemented with 1 mM CaCl₂), and eluted with 100 mM Glycine-HCl (pH 2.3), 1 mM CaCl₂. The eluate was immediately neutralized with 1M Tris pH 9.0 (1/15, v/v). Protein fractions were pooled and dialyzed in TBS/Ca overnight at 4° C. Protein concentration was determined by the Bradford method (Bio-Rad, Hercules, Calif.) using bovine IgG as standard, and protein purity was evaluated by SDS-PAGE.

Biotinylation

E-cadherin-IgG fusion protein was dialyzed in 10 mM Na-borate (pH 8.4), 0.5 mM CaCl₂ for overnight at 4° C., and labeled with aminohexanoyl-biotin-N-hydroxysuccinimide (AH-BNHS, Zymed, South San Francisco, Calif.) at a ratio of 1:10 (AH-BNHS/protein, w/w) for 1 hour at room temperature. The labeled protein was dialyzed in TBS (20 mM Tris-HCL, pH 7.5, 150 mM NaCl) supplemented with 1 mM CaCl₂ at 4° C. Protein concentration was determined using the Bradford method (Bio-Rad).

Soluble Recombinant αEβ7 Protein (ts αEβ7.coil)

A nucleic acid encoding the extracellular domain of integrin αE chain (amino acid residues 1-1105 of SEQ ID NO: 2) was fused with a nucleic acid encoding a 30 amino acid acidic peptide (AQLEKELQALEKENAQLEWELQALEKELAQ, SEQ ID NO: 39) to create a construct designated αE-acid. A nucleic acid encoding the extracellular domain of the β7 subunit (amino acid residues 1-707 of SEQ ID NO: 38) was fused with a nucleic acid encoding a 30 amino acid basic peptide (AQLKKKLQALKKKNAQLKWKLQALKKKLAQ, SEQ ID NO: 40) to create a construct designated β7-base. When expressed, the acidic and basic peptides form a heterodimeric coiled coil. (See, Lu et al, J. Biol. Chem. 276:14642-14648 (2001); O'Shea et al., Curr. Biol. 3:658-667 (1993).) A nucleic acid encoding a linker of six amino acid residues (GGSTGG, SEQ ID NO: 41) was inserted into both constructs (between the αE sequence and the acidic peptide, as well as between the β7 and the basic peptide). The αE and β7 fusion constructs were separately cloned into the expression vector AprM8 (see, Lu and Springer, J. Immunol. 159:268-278 (1997)), and sequences were confirmed by DNA sequencing.

Expression and Purification

The αE-acid and β7-base constructs were transiently transfected into 293T cells and co-expressed to produce a soluble αEβ7. The secretion of soluble αEβ7 heterodimer (ts αEβ7.coil) by the transfected cells was confirmed by ELISA and immunoprecipitation using several antibodies that bound the αE or β7 subunit. For large-scale purification, 30-35 10 cm-plates of 293T cells were co-transfected with αE-acid and β7-base constructs, and culture supernatant was collected as described above.

ts αEP7.coil was purified by column chromatography using an anti-β7 antibody (mAb 6F7; Millennium Pharmaceuticals Inc., Cambridge, Mass.) affinity column. mAb 6F7 was covalently coupled to CNBr-activated SEPHAROSE 4B beads (beaded agarose, Pharmacia). Culture supernatant containing ts αEβP7.coil was applied to the column at 4° C. The column was washed with TBS (20 mM Tris-base, pH 7.5, 150 mM NaCl), 1 mM CaCl₂ and 1 mM MgCl₂ in cold, and eluted in 50 mM triethylamine (TEA), pH 11.5, 150 mM NaCl, 1 mM CaCl₂ and 1 mM MgCl₂. The eluate was immediately neutralized with 1 mM Tris-HCl, pH 6.8, 5 mM CaCl₂ and 5 mM MgCl₂ (1/10, v/v). Protein fractions were pooled, concentrated using CENTRICON-30 membrane concentrator (Millipore, Bedford, Mass.), and buffer was changed to TBS, pH 7.5 containing 1 mM CaCl₂ and 1 mM MgCl₂. The protein concentration was determined, and the purified sample was aliquoted and stored at −70° C. The purity of ts αEβ7.coil protein was about 90% as judged by SDS-PAGE and silver staining.

Transfectants

L1.2 cells (murine B lymphoma cell line) were cultured in RPMI/10% FetalClone (Hyclone). K562 cells were maintained in RPMI/10%FBS (Gibco). For stable expression of αEβ7, 20 μg of αE full length cDNA (SEQ ID NO: 1) in AprM8 and 20 μg of β7 cDNA (SEQ ID NO: 37) in AprM8 were linearized and cotransfected with 1 μg linear PEFpuro (see, Lu and Springer, J. Immunol. 159:268-278 (1997)), which contains puromycin selection marker, by electroporation at 250 V, 960 μF using 0.4 cm cuvette. 48 hours post transfection, cells were collected, and resuspended in culture medium supplemented with 2 μg/ml or 4 μg/ml puromycin for L1.2 transfectants and K562 transfectants, respectively. Cells were subsquently subcloned in 96-well plates. Clones of transfectants were tested for αEβ7 cell surface expression by staining with mouse anti-αE and anti-β7 antibodies. Selected clones were subcloned again.

Mouse anti-αE mAb αE7.1 was described previously. (Russel, G. J. et al., Eur. J. Immunol. 24:2832-2841 (1994).) 293T cells (human embryonic kidney epithelial cell line) were maintained in Dulbecco's Modified Eagles Medium/10% FBS (Gibco), supplemented with essential amino acids and sodium pyruvate.

Generation of αEβ7-Specific Human Antibodies

Human monoclonal antibodies, mAb 3G6, mAb 5E4 and mAb 8D5, were generated using human-antibody transgenic mice that express human immunoglobulin genes. mAb 5E4 and mAb 3G6 were produced using HUMAB mice (MEDAREX, Princeton, N.J.), and mAb 8D5 was produced using XENOMOUSE mice (Abgenix, Fremont, Calif.). The same immunization, fusion and antibody screening protocols were used to produce human monoclonal antibody 3G6, human monoclonal antibody 5E4 and human monoclonal antibody 8D5.

Immunization

L1.2 transfectants that express human αEβ7 were treated with mitomycin C at 100 μg/ml for 30 minutes at 37° C. Cells were washed twice with phosphate buffered saline (PBS), and resuspended at 2×10⁷ cells/ml in PBS. Mice were injected with about 0.5 ml of the resulting cell suspension (intraperitonial injection (IP), 10⁷ cells/mouse/injection) at about two week intervals. After 4 IP injections, mice were boosted with purified recombinant αEβ7 protein (ts αEβ7.coil)(15 μg/mouse, intravenous (IV) injection). 4 days after the IV boost, mice were tested for αEβ7-specific human IgG response in the serum. Spleens from positive mice were used for fusion.

Titration of αEβ7-Specific Human IgG

A sandwich ELISA was used to titrate mouse sera containing human IgG antibodies that bind αEβ7 integrin. ELISA plates were coated with 15 μg/ml mouse anti-β7 mAb 6F7 (50 μl/well) at 37° C. for 2 hours. The plates were then washed with PBS and incubated with 50 μl culture supernatant containing recombinant αEβ7 protein overnight at 4° C. The plate was washed twice with PBS, and incubated with mouse anti-serum at various dilutions in PBS at 37° C., for 1 hour. Then, the plates were washed twice, and the plate was incubated with HRP-conjugated goat anti-human IgG at 37° C. for 1 hour. The plates were then washed again and human antibodies that bound αEβ7 were detected by addition of peroxidase substrate, and absorbance was read on an ELISA reader at 410 nM wavelength.

Hybridomas that Produce Antibodies that Bind αEβ7

Spleens were removed from mice that produced anti-αEβ7 antibodies and splenocytes were fused with myeloma cells (SP2/0) to produce hybridomas.

Hybridomas were Screened for Production of Anti-αEβ7 Antibodies using a Flow Cytometry Assay and an ELISA.

L1.2 αEβ7 transfectants or untransfected cells (negative control) were collected by centrifugation, and resuspended to 10⁷ cells/ml in PBS/5% FBS. 50 μl of cell suspension (5×10⁵ cells) was incubated with 50 μl hybridoma supernatant in a 96-well plate for 30 minutes on ice. The cells were washed once with PBS/5% FBS, and incubated with FITC-conjugated anti-human IgG or IgM for 30 minutes on ice. The cells were washed again, resuspended in PBS, and antibody binding was measured by flow cytometry using a FACS instrument. Hybridoma supernatants that stained L1.2 αEβ7 transfectants but not the untransfected parental L1.2 cells were saved and screened further by αEβ7-specific ELISA. The protocol for the ELISA was identical to the ELISA described above except that 50 μl hybridoma supernatant was used instead of diluted serum. Positive hybridomas were further tested for αE specificity.

Screen for αE-Specific Antibodies

FACS staining of K562 transfectants that express either αEβ7 or α4β7 integrin was used. FACs staining protocol was the same as described above. Hybridomas that stained αEβ7 transfectants but not α4β7 transfectants were selected as producing αE-specific antibody. αE-specific hybridomas were further subcloned at least twice by limiting dilution.

Assays for Selecting Antibodies that Inhibit Binding of αEβP7 to E-cadherin

Cell Adhesion Assay.

ELISA plates were coated with 100 ng/well E-cadherin-IgG fusion protein in TBS (20 mM Tris, 140 mM NaCl, pH 9)/1 mM CaCl₂ overnight at 4° C. Plates were washed with wash buffer (HBSS/1 mM CaCl₂), and blocked with HBSS/1 mM CaCl₂/2% BSA for one hour at 37° C. After blocking, plates were washed twice with wash buffer. K562 transfectants at log growth stage were collected, washed once in HBSS/0.2% BSA/1 mM CaCl₂/1 mM MgCl₂, and resuspended to 4×10⁶ cells/mL in the same buffer. Cells were labeled with the fluorecent dye BCECF-AM (Molecular Probes, 4 μg/ml final concentration) for 15 minutes at 3° C. Labeled cells were washed twice, and resuspended in assay buffer (HBSS/0.2% BSA/1 mM CaCl₂/1 mM MgCl₂/1 mM MnCl₂) to 8×10⁵ cells/mL. 50 μl of the cell suspension was added to the E-cadherin-IgG coated well (4×10⁴ cells/well), and mixed with 50 μl assay buffer containing antibodies with desired concentration, or isotype-matched control antibody. The plate was then incubated at room temperature for 1 hour. The fluorescence content in each well was read on a Fluorescent Concentration Analyser (IDEXX, Westbrook, Me.) before and after three washes with HBSS/0.5 mM CaCl₂/0.5 mM MgCl₂/0.5 mM MnCl₂ using a Microplate Autowasher (Bio-Tek instruments, Winooski, Vt.). The Microplate Autowasher was programmed with parameters: 250 μl wash volume, 1× wash cycle, 0 soak time, and aspiration tube depth of 70. The bound cells (after washes) were expressed as a percentage of total input cells (before washes) in each well. Each sample was set up in triplicate wells.

The effect of activation of αEβ7 integrin by divalent cations was evaluated in cell adhesion assays using transfected K562 cells that expressed αEβ7 integrin. The transfected K562 cells were fluorescently labeled and added to assay wells that were coated with E-cadherin-IgG fusion protein (100 ng/well). The assay media contained CaCl₂ and MgCl₂ (1 mM each; Ca+Mg); CaCl₂, MgCl₂ and MnCl₂ (1 mM each; Ca+Mg+Mn); or the divalent cation chelating agent EDTA (5 mM). The fluorescently labeled cells were allowed to adhere to the plate-bound E-cadherin-IgG fusion protein, unbound cells were washed away and bound cells are detected by measuring fluorescence. Cell binding was enhanced in media that contained MnCl₂ and inhibited in media that contained EDTA (relative to media that contained media contained CaCl₂ and MgCl₂).

Cell-Free αEβ7/E-Cadherin Binding Assay.

Purified recombinant αEβ7 (ts αEβ7.coil) was diluted to 5 μg/ml in TBS, pH 8/Ca+Mg (20 mM Tris, pH 8, 140 mM NaCl, 1 mM CaCl₂ and 1 mM MgCl₂), and 50 μl was used to coat each well of 96-well ELISA plate overnight at 4° C. The plate was washed in wash buffer (20 mM Tris, pH 7.5, 140 mM NaCl, 1 mM CaCl₂ and 1 mM MgCl₂), and blocked with 300 μl/well blocking buffer (20 mM Tris, pH 7.5, 140 mM NaCl, 1 mM CaCl₂ and 1 mM MgCl₂, 2% BSA) for 2 hours at 37° C. 25 μl of biotin-labeled E-cadherin-IgG fusion protein diluted to 20 μg/ml in assay buffer (20 mM Tris, pH 7.5, 140 mM NaCl, 1 mM CaCl₂, 1 mM MgCl₂, 1 mM MnCl₂, and 1%BSA) was added to each αEβ7-coated wells, and mixed with 25 μl assay buffer containing test antibodies at desired concentration, or isotype-matched control antibody. The plate was then incubated for 90 minutes at 37° C. The plate was then washed twice with wash buffer, and 50 μl HRP-streptavidin (1:1000 dilution in assay buffer) was added to each well, and the plate was incubated for 1 hour at 37° C. Color was developed by adding substrate buffer (ABTS substrate for HRP, Zymed), and absorbance was read on an ELISA plate reader (410 nm).

The effect of activation of αEβ7 integrin by divalent cations was evaluated in this cell-free adhesion assays using assay buffer that contained CaCl₂ and MgCl₂ (1 mM each; Ca+Mg); CaCl₂, MgCl₂ and MnCl₂ (1 mM each; Ca+Mg+Mn); or the divalent cation chelating agent EDTA (5 mM).

Conversion of 5E4 (IgM), 3G6 (IgM) and 8D5 (IgG2) to Human IgG1-FcRmut Isotype

RNA was prepared from 1×10⁷ hybridoma cells using QIAGEN RNEASY RNA isolation kit (QIAGEN, Valencia, Calif.) according to manufacturer's instruction. cDNA was synthesized, and variable regions of light and heavy chains were cloned out by PCR. VL(kappa) regions were cloned using human IG-PRIMER oligonucleotide primers (Novagen, Madison, Wis.), and VH regions were made using synthetic primers AB85-89 (SEQ ID NOS: 46-50) and AB90 (MEDAREX, Princeton, N.J.; SEQ ID NO: 51) for hybridomas 5E4 and 3G6 or synthetic primers pHuVH1-7 (SEQ ID NOS: 52-58) and NHuIgG2p3 (SEQ ID NO: 59) for hybridoma 8D5.

PCR fragments were cloned into PCR2.1-TOPO vector using a TOPO cloning kit (Invitrogen, Carlsbad, Calif.), and 6-8 clones from each PCR reaction were sequenced to determine consensus of variable region sequences. The variable regions were subsequently isolated from PCR2.1-TOPO vectors by PCR using primers with restriction enzyme sites incorporated at both ends for subcloning (MfeI and BlpI sites for VH; EcoRI and BsiWI for 5E4 VL and 3G6 VL; or PpuMI and BsiWI for 8D5 VL). Primers p3G6VH5 (SEQ ID NO: 60) and pAEB7VH3 (SEQ ID NO: 62) were used for the 3G6 VH, primers pAEB7VH5 (SEQ ID NO: 61) and pAEB7VH3 (SEQ ID NO: 62) were used for the 5E4 VH, primers pAEB7VK5 (SEQ ID NO: 63) and pAEB7VK3 (SEQ ID NO: 64) were used for the 3G6 and 5E4 VLs. The primers for the VL of 5E4 and 3G6 include the VL leader sequence whereas all other primers allow cloning into antibody expression vectors that contain VH and VL leaders.

The PCR products encoding the VH of either 5E4 or 3G6 were separately subcloned into the MfeI and BlpI sites of pLKTOK30. pLKTOK30 is based on the pCDNA3 vector with the CMV promoter replaced with the EF-1a promoter. pLKTOK30 contains sequences encoding a VH leader and a human IgG1 constant region that are separated by the desired cloning sites. The human IgG1 constant region encoded by this vector contains the Leu 235 to Ala 235 and Gly 237 to Ala 237 mutations that interfere with the antibody binding to Fc receptors (human IgG1-FcR mut region). The MfeI site is within the bases VH3-4 and the BlpI site is at the junction of VH and CH.

The PCR products encoding the VL of either 5E4 or 3G6 were separately subcloned into the EcoRI and BsiWI sites of pLKTOK25. pLKTOK25 has a similar structure to pLKTOK30 with the exception that it contains a sequence that encodes a human kappa constant region instead of a human IgG1 constant region and does not contain a sequence encoding a leader. In this vector, the Kozak sequence and sequence encoding a VL leader are included with the adapted VL gene fragments.

The heavy and light chain containing vectors for each antibody (5E4 or 3G6) were cotransfected in 293T cells to evaluate IgG1 production. When production of functional antibody was confirmed, the heavy chain including the promoter region was excised from TOK30 vector with HindIII and XbaI and ligated into the same sites (HindIII and XbaI) of the light chain containing TOK25 vector to generate a single IgG1 expression vector. The single IgG1 expression vectors were used to make stable CHO cells expressing either 5E4 or 3G6 antibody as described below.

The VH and VL of 8D5 were adapted and cloned into the antibody expression vector pLKTOK59 using PCR. Vector pLKTOK59, like pLKTOK30, is based on the pCDNA3 vector. However, pLKTOK59 contains two EF-1a promoters, one of which drives expression of the heavy chain while the other drives expression of the light chain. The 8D5 VH gene was adapted by PCR using synthetic primers p8D5VH5 (SEQ ID NO: 65) and p8D5VH3 (SEQ ID NO: 66) to add the cloning sites MfeI and BlpI and cloned between the VH leader and Human IgG1-FcRmut region of pLKTOK59D. The 8D5 VL gene was adapted by PCR using synthetic primers p8D5VK5 (SEQ ID NO: 67) and p8D5VK3 (SEQ ID NO: 68) to add the cloning sites PpuMI and BsiWI and cloned between the VL leader and human kappa constant region of pLKTOK59D-8D5-VH to create pLKTOK59D-8D5-VHVK.

Expression of Converted IgG1 Antibodies and Preparation of Stable CHO Cells

Medium scale production of 3G6 (IgG1) and 5E4 (IgG1) was done in 293T cells using calcium phosphate transfection. 10 μg of each heavy and light chain expression vector were used to transfect one 10 cm plate of 293T cells. 7-11 hour post-transfection, the culture medium was changed to media supplemented with 10% ultra low IgG FBS (Gibco). The transfected cells were cultured and the culture supernatant (10 mL) was collected daily for three days. A total of about 900 mL supernatant for each antibody was collected.

Stable CHO cell lines were generated using the single IgG1 expression vectors described above that contain both heavy and light chains of the converted IgG1 antibodies. CHO (DG44) stable transfection was performed using FUGENE non-liposomal lipid transfection (Boehringer Mannheim) according to manufacturer's instruction. 2 days after transfection, CHO cells were collected, resuspended in selection medium (alphaMEM, 10% Hyclone serum, 800 mg/L G418), and subcloned into 96 well plates. Several stable CHO clones that secreted IgG1 antibodies were selected and the high producers were subcloned again. The yield of IgG1 production by the stable CHO lines was determined by ELISA assay using human IgG1 as standard, and the functional activity of the IgG1 antibodies was determined by binding to αEβ7 transfectants and blocking αEβ7 interaction with E-cadherin.

The “3G6 CHO stable cell line,” also referred to as CHO 3G6 C1.2D6, which produces an IgG1 form of mAb 3G6 was deposited on Apr. 3, 2002, on behalf of Millennium Pharmaceuticals Inc., 75 Sidney Street, Cambridge, Mass., 02139, USA, at the American Type Culture Collection, 10801 University Boulevard, Manassas, Va. 20110, U.S.A., under Accession No. PTA-4204.

The “5E4 CHO stable cell line,” also referred to as CHO 5G4 A1.2C12, which produces an IgG1 form of mAb 5E4 was deposited on Apr. 3, 2002, on behalf of Millennium Pharmaceuticals Inc., 75 Sidney Street, Cambridge, Mass., 02139, USA, at the American Type Culture Collection, 10801 University Boulevard, Manassas, Va. 20110, U.S.A., under Accession No. PTA-4205.

Purification of IgG Antibodies

The converted 3G6 (IgG1) and 5E4 (IgG1) antibodies were purified from culture supernatant of transiently transfected 293T cells, and the 8D6 IgG2 antibody was purified from hybridoma supernatant. Protein A agarose (Gibco) columns were used to purify both IgG1 and IgG2 antibodies. Briefly, antibody-containing supernatants were run through the Protein A column overnight at 4° C. at a slow flow rate. Then, the column was washed with TBS (20 mM Tris-HCl pH 7.5, 140 mM NaCl) at 4° C. and eluted with 100 mM Glycine-HCl pH 2.3. The eluate was immediately neutralized with 1M Tris-HCl pH 9.0 (1/15 v/v). Fractions were pooled and dialyzed in PBS at 4° C. Antibody concentration was determined by the Bradford method (Bio-Rad) using bovine IgG as standard. Antibody purity was analyzed by SDS-PAGE.

Determination of Half Saturation Concentration of mAb Binding to αEβ7 transfctants

Purified antibodies were serially diluted in PBS/5% FBS. FACS staining using the diluted antibodies and K562 transfectants that express αEβ7 on the cell surface was performed as described above. FITC-conjugated anti-human IgG1 or FITC-conjugated anti-human IgG2 was used as secondary antibody. The degree of staining (mean fluorescence intensity) detected was plotted against the concentration of antibody used (μg/ml), and half saturation concentrations were determined using the plot. To determine Mn²⁺ effect on antibody binding, 1 mM MnCl₂ (final concentration) was included in the staining buffer in some studies.

Antibody IC50 Determination

IC50 was determined using the cell adhesion assay and cell-free αEβ7/E-cadherin binding assays described above. The percentage of input cells that bound αEβ7 (cell adhesion assay) or amount of E-cadherin-IgG fusion that bound αEβ7 (measured by absorbance in the cell-free binding assay) was plotted against the concentration of antibody used (μg/ml), an inhibition curve was drawn, and IC50 values were determined using the curve.

Culture of Human Peripheral Blood Lymphocytes

Human PBL were purified from fresh whole blood using standard Histopaque gradient centrifugation, and cultured at 1×10⁶ cells per mL in RPMI 1640 with 10% FBS, supplemented with TGF-β1 and IL-2 to increase αEβ7 expression on the surface. After culture for 10-15 days, cells were collected for FACS staining as described above.

Epitope Mapping

Construction and Expression of αE I-domain-Fc Fusion Protein.

A nucleic acid encoding the αE chain I-domain (amino acids 161-379 of SEQ ID NO: 2) was isolated from full-length αE cDNA (SEQ ID NO: 1) by PCR using synthetic primers aEXID(5) (tcggatccgctctggagaaggaggag, SEQ ID NO: 69) and aEIDS(3) (gcgaattcaagggcgtctccaaccgt, SEQ ID NO: 70). This nucleic acid was joined in-frame with a nucleic acids encoding the αE secretion signal sequence (amino acids −18 to −1 of SEQ ID NO: 2) and a human IgG1 Fc region that contained mutations to reduce binding to Fc receptor, to produce a construct encoding the αE I-domain Fc fusion protein.

The fusion construct encoded a fusion protein that contains GlySer at the amino-terminus of the I domain and GluPhe between the I domain and the Fc region sequences. The I domain encoded by the fusion construct also includes a portion of the X domain. These X domain sequences ensure proper folding and secretion of the I domain fusion protein.

The αE I-domain-Fc fusion construct was cloned in vector pCDEF3, which was transiently expressed in 293T cells. Culture supernatant that contained the fusion protein was collected as described above.

Binding of mAb to αE I-domain-Fc Fusion Protein

An ELISA assay was established to evaluate secretion and folding of the I-domain fusion protein using mouse antibodies previously mapped to the αE I-domain. (Higgins, J. M. G. et al., J. Biol. Chem. 275:25652-25664 (2000).) In this assay, anti-human IgG was immobilized to capture the αE I-domain-Fc fusion protein from the supernatant of 293T transfectants, and binding of two mouse antibodies, αE7.1 and HML-1, was tested. (Higgins, J. M. G. et al., J. Biol. Chem. 275:25652-25664 (2000).) The two mouse mAb bound to the αE I-domain-Fc fusion protein. Binding of human antibodies was determined using similar assay conditions. Goat anti-human IgG (15 μg/ml in 20 mM Tris, pH9, 140 mM NaCl) was used to coat 96 well ELISA plate overnight at 4° C. The plate was blocked with 2%BSA and incubated with 50 μl culture supernatant of 293T transfected with the αE I-domain-Fc fusion construct or with vector alone (control) for 1 hour at 37° C. After washing, the plate was incubated with 50 μl of hybridoma supernatant of 5E4 IgM, 3G6 IgM or 8D5 IgG2. Binding of IgM antibody to the captured αE I-domain-Fc fusion protein was detected by HRP-conjugated anti-human IgM or anti-human IgG2.

Antibody Competition Assay (Cytometry Assay)

5×10⁵ K562 transfectants expressing αEβ7 were incubated with mouse mAb αE7.1 (15 μg/ml)(Russel, G. J. et al., Eur. J. Immunol. 24:2832-2841 (1994)), human mAb 5E4 (IgM hybridoma supernatant), human 3G6 (IgM hybridoma supernatant), or medium control on ice for 30 minutes. Then, the cells were washed and incubated with human mAb 8D5 (IgG2, 15 μg/ml) for 30 minutes on ice. Cells were then washed twice and incubated with FITC-anti-human IgG, and analyzed by fluorescence flow cytometry.

Fine Specificity

The fine specificity of mAb 5E4 was determined using a panel of transfected K562 cells that expressed various mutant αEβ7 integrins and detecting antibody binding to the transfectants by flow cytometry. The mutants proteins and methods used have been previously described in Higgins, J. M. G. et al., J. Biol. Chem. 275:25652-25664 (2000). The mutant αEβ7 integrins used contained the following mutations in the αE chain: R159S/R160S; ΔE163-E180; ΔE176; D190A; G193A; D199A; R202A/D205A; G230A/V231A; D240A; F298A; P311H/E345A/T346A; E325A; and Y354W. (See, Higgins, J. M. G. et al., J. Biol. Chem. 275:25652-25664 (2000).)

### Results

Hybridomas that produce human antibodies which bind αEβ7 integrin were produced, and the antibodies produced by three of the hybridomas were characterized. The supernatants of hybridomas 3G6 (which produces an IgM), 5E4 (which produces an IgM) and 8D5 (which produces an IgG2) were tested for αEβ7 binding specificity by flow cytometry. mAb 3G6 (IgG1), mAb 5E4 (IgG1) and mAb 8D5 each bound transfected L1.2 cells and transfected K562 cells that expressed αEβ7 integrin, but none of these antibodies bound transfected K562 cells that expressed α4β7 integrin, indicating that the mAbs have binding specificity for integrin αE chain. Each mAb (mAb 3G6 (IgM), mAb 3G6 (IgG1), mAb 5E4 (IgM), mAb 5E4 (IgG1) and mAb 8D5) inhibited binding of αEβ7 integrin to its ligand E-cadherin using an in vitro cell adhesion assay and also inhibited binding of soluble E-cadherin-Fc to immobilized αEβ7 integrin in a cell free adhesion assay.

The variable regions of mAb 3G6, mAb 5E4 and mAb 8D5 were cloned and constructs encoding these antibodies with a human IgG1 constant region were produced. The IgG1 versions of mAb 3G6 (IgG1) and mAb 5E4 (IgG1) were used in some of the studies described herein.

The concentration of mAb 3G6 (IgG1), mAb 5E4 (IgG1) and mAb 8D5 that resulted in half saturation of antibody binding sites on transfected K562 cells that expressed αEβ7 was determined using flow cytometry. mAb 3G6 (IgG1) and mAb 5E4 (IgG1) both had a half saturation concentration of 1 μg/mL, while mAb 3G6 had a half saturation concentration of 2.5 μg/mL. The concentrations of antibody that inhibited binding in the cell adhesion assay and the cell free binding assay (IC50) were also determined for mAb 3G6 (IgG1), mAb 5E4 (IgG1) and mAb 8D5. The IC50 for mAb 3G6 (IgG1) was about 2.04 μg/mL (13.4 nM) in the cell adhesion assay, and about 0.089 μg/mL (0.59 nM) in the cell free assay. The IC50 for mAb 5E4 (IgG1) was about 1.29 μg/mL (8.5 nM) in the cell adhesion assay, and about 1.02 μg/mL (6.7 nM) in the cell free assay. The IC50 for mAb 8D5 (IgG1) was about 0.715 μg/mL (4.7 nM) in the cell adhesion assay, and about 0.197 μg/mL (1.30 nM) in the cell free assay.

Integrin molecules, such as αEβ7, bind their ligands with high affinity when activated by, for example, divalent cations (e.g., Mn²⁺). The results of cellular binding studies revealed that recombinant αEβ7 expressed on transfected K562 cells is activated by divalent cation ions, particularly Mn²⁺, and binding to immobilized E-cadherin is enhanced under conditions where Mn²⁺ is present. Similar results were obtained in studies in which transfected K562 cells were stained with Biotin-E-cadherin-IgG. The result of the cellular binding assay are presented in Table 1, and the results of the staining assay are presented in Table 2.

TABLE 1

| Assay Buffer | % cells bound to immobilized E-cadherin |
|---|---|
| Ca²⁺ and Mg²⁺ (1 mM each) | ˜40% |
| Ca²⁺ and Mg²⁺ and Mn²⁺ | ˜60% |
| (1 mM each) | |
| EDTA (5 mM) | <10% |

TABLE 2

| Biotin-E- | | Binding |
|---|---|---|
| cadhering-IgG | | (mean fluorescence |
| used (μg/ml) | Staining Buffer | intensity) |
| 2.5 | Ca²⁺ and Mg²⁺ (1 mM each) | <25 |
| | Ca²⁺ and Mg²⁺ and Mn²⁺ | 50-75 |
| | (1 mM each) | |
| 5 | Ca²⁺ and Mg²⁺ (1 mM each) | 25-50 |
| | Ca²⁺ and Mg²⁺ and Mn²⁺ | ˜100 |
| | (1 mM each) | |
| 10 | Ca²⁺ and Mg²⁺ (1 mM each) | ˜75 |
| | Ca²⁺ and Mg²⁺ and Mn²⁺ | 150-175 |
| | (1 mM each) | |

To determine whether mAbs 3G6, 5E4 or 8D5 bound an activation-induced epitope, αEβ7 expressing K562 transfectants were stained with antibodies using a buffer that contained Mn²⁺ and using a buffer that did not contain MW²⁺, and antibody binding was detected by fluorescence flow cytometry. The results of these studies demonstrated that binding of mAb 3G6 (IgG1) was enhanced in the presence of Mn²⁺, but that the binding of mAb 5E4 (IgG1), mAb 8D5 and mAb αE7.1 to αEβ7 integrin on the K562 transfectants was about equivalent in buffers that contained or did not contain Mn²⁺. The results show that mAb 3G6 (IgG1) preferentially bound Mn²⁺ activated αEβ7 integrin on transfected K562 cells.

These results were confirmed in antibody binding studies using primary human peripheral blood mononuclear cells. The human PBMC were cultured in IL-2 and TGF-β for 10-15 days, which increased the percentage of CD3+αE+cells to about 30-40%. The cells were then stained with mAb 3G6 (IgG1), mAb 5E4 (IgG1), mAb 8D5 or mAb αE7.1 using a buffer that contained Mn²⁺ and using a buffer that did not contain Mn²⁺, and antibody binding to CD3+ cells was detected by fluorescence flow cytometry. As with transfected K563 cells, binding of mAb 3G6 (IgG1) was enhanced in the presence of Mn²⁺ (positive cells in buffer without Mn²⁺, <5%; positive cells in buffer with Mn²⁺, ˜20%), whereas binding of mAb 5E4 (IgG1), mAb 8D5 and mAb αE7.1 was about equivalent in buffers that contained or did not contain Mn²⁺. These results demonstrate that integrin αE chain can adopt an activated conformation and that mAb 3G6 preferentially binds an activation-induced epitope on integrin αE chain.

Epitopic specificity of the mAbs was studied further using an αE I-domain-Fc fusion protein, a panel of transfected K562 cells that expressed various mutant αEβ7 integrins (see, Higgins, J. M. et al., J. Biol. Chem. 275:25652-25664 (2000)), and antibody blocking studies using transfected K562 cells that expressed αEβ7. Mabs 3G6 (IgM), 5E4 (IgM) and 8D5 (hybridoma culture supernatants) each bound αE I-domain-Fc fusion protein coated wells in the ELISA, but binding above control levels was not detected in wells coated with supernatants from mock transfected 293T cells that did not produce the αE I-domain-Fc fusion protein, demonstrating that each antibody binds an epitope that includes amino acids in the I domain of E-cadherin.

The fine specificity of mAb 5E4 was examined using transfected K562 cells that expressed αEβ7 integrin or mutated version of αEβ7 integrins, and antibody binding was detected by flow cytometry. The results are shown in Table 3. Antibody binding was inhibited by deletion of amino acid residues 163-180 (ΔE163-E180; amino acid residues 163-180 of SEQ ID NO: 2), which are in the X domain of integrin αE chain, and was essentially abrogated by mutation of amino acid residue 298 (amino acid residue 298 of SEQ ID NO:2), which is in the I-domain, from Phenylalanine to Alanine (F298A).

TABLE 3

| | % Control | | % Control |
|---|---|---|---|
| αE Mutation | Staining | αE Mutation | Staining |
| none | 100% | R202A/D205A | 75-100% |
| R159S/R160S | 75-100% | G230A/V231A | 75-100% |
| ΔE163-E180 | ˜25% | D240A | 75-100% |
| ΔE176 | 75-100% | F298A | no binding detected |
| D190A | 50-75% | P311H/E345A/T346A | 50-75% |
| G193A | 75-100% | E325A | 75-100% |
| D199A | 75-100% | Y354W | ˜75% |

The fine specificity of several anti-αE antibodies have been evaluated using this method and the mutant αEβ7 integrins. For most antibodies tested, binding to the ΔE163-E180 mutant is inhibited relative to binding to un-mutated αEβ7. Thus, this inhibition appears to be nonspecific and may be the result of instability of the mutant and/or proteolytic degradation. In contrast, antibody binding was essentially abrogated by mutation of amino acid 298, which is in the I-domain, from Phenylalanine to Alanine (F298A), indicating that epitope bound by mAb 5E4 includes Phe298.

The results of flow cytometry based antibody blocking studies revealed that pre-incubating transfected K562 cells that expressed αEβ7 integrin with mAb αE7.1 partially inhibited binding of mAb 8D5, indicating that these antibodies may bind adjacent or overlapping epitopes. However, the inhibition could be the result of steric interference. Binding of mAb 8D5 was not significantly inhibited when the transfected cells were pre-incubated with mAb 5E4 (IgM) or mAb 3G6 (IgM), demonstrating that mAbs 3G6, 5E4 and 8D5 bind distinct epitopes.

While this invention has been particularly shown and described with references to preferred embodiments thereof, it will be understood by those skilled in the art that various changes in form and details may be made therein without departing from the scope of the invention encompassed by the appended claims.

What is claimed is:

1. An antibody or antigen-binding fragment thereof which binds an activated αE integrin, wherein said antibody or antigen-binding fragment specifically binds an activation-induced epitope on integrin αE chain (CD103).

2. The antibody or antigen-binding fragment of claim 1 wherein said activation-induced epitope is induced by activation with a divalent cation.

3. The antibody or antigen-binding fragment of claim 1 wherein said activation-induced epitope is in the I domain of integrin αE chain (CD103).

4. The antibody or antigen-binding fragment of claim 1 wherein said antibody or antigen-binding fragment:
a) inhibits binding of ligand to said αE integrin;
b) inhibits αE integrin-mediated adhesion of a cell expressing said αE integrin to epithelial cells or endothelial cells; or
c) competitively inhibits binding of monoclonal antibody 3G6 to said αE integrin.

5. The antibody or antigen-binding fragment of claim 4 wherein said αE integrin is αEβ7 integrin.

6. The antibody or antigen-binding fragment of claim 4 wherein said antibody or antigen-binding fragment inhibits binding of ligand to said αE integrin, and said αE integrin is αEβ7 integrin and said ligand is E-cadherin.

7. The antibody or antigen-binding fragment of claim 1 wherein said antibody or antigen-binding fragment is selected from the group consisting of:
a) a human antibody or an antigen-binding fragment of a human antibody;
b) a humanized antibody or an antigen-binding fragment of a humanized antibody; and
c) a chimeric antibody or an antigen-binding fragment of a chimeric antibody.

8. An antibody or antigen-binding fragment thereof which binds an αE integrin and inhibits binding of ligand to said αE integrin, wherein said antibody or antigen-binding fragment comprises at least one heavy chain complementarity determining region (HCDR1, HCDR2 and/or HCDR3) comprising an amino acid sequence selected from the group consisting of:
HCDR1 SEQ ID NO: 5 or SEQ ID NO: 5 wherein one or two amino acids are conservatively substituted;
HCDR2 SEQ ID NO: 6 or SEQ ID NO: 6 wherein one or two amino acids are conservatively substituted; and
HCDR3 SEQ ID NO: 7 or SEQ ID NO: 7 wherein one or two amino acids are conservatively substituted; and
at least one light chain complementarity determining region (LCDR1, LCDR2 and/or LCDR3) comprising an amino acid sequence selected from the group consisting of:
LCDR1 SEQ ID NO: 10 or SEQ ID NO: 10 wherein one or two amino acids are conservatively substituted;
LCDR2 SEQ ID NO: 11 or SEQ ID NO: 11 wherein one or two amino acids are conservatively substituted; and
LCDR3 SEQ ID NO: 12 or SEQ ID NO: 12 wherein one or two amino acids are conservatively substituted.

9. The antibody or antigen-binding fragment of claim 8 wherein said antibody or antigen-binding fragment selectively binds an activation-induced epitope on integrin αE chain (CD103).

10. An immunoglobulin heavy chain or antigen-binding portion thereof comprising three heavy chain complementarity determining regions (HCDR1, HCDR2 and HCDR3) comprising the following amino acid sequences:
HCDR1 SEQ ID NO: 5 or SEQ ID NO: 5 wherein one or two amino acids are conservatively substituted;
HCDR2 SEQ ID NO: 6 or SEQ ID NO: 6 wherein one or two amino acids are conservatively substituted; and
HCDR3 SEQ ID NO: 7 or SEQ ID NO: 7 wherein one or two amino acids are conservatively substituted,
wherein an antibody comprising said heavy chain or antigen-binding portion thereof and a complementary light chain or antigen-binding portion of a complementary light chain binds an αE integrin.

11. An immunoglobulin light chain or antigen-binding portion thereof comprising three light chain complementarity determining regions (LCDR1, LCDR2 and LCDR3) comprising the following amino acid sequences:
LCDR1 SEQ ID NO: 10 or SEQ ID NO: 10 wherein one or two amino acids are conservatively substituted;
LCDR2 SEQ ID NO: 11 or SEQ ID NO: 11 wherein one or two amino acids are conservatively substituted; and
LCDR3 SEQ ID NO: 12 or SEQ ID NO: 12 wherein one or two amino acids are conservatively substituted,
wherein an antibody comprising said light chain or antigen-binding portion thereof and a complementary heavy chain or antigen-binding portion of a complementary heavy chain binds an αE integrin.

12. An antibody or antigen-binding fragment thereof which binds an αE integrin and inhibits binding of ligand to said αE integrin, wherein
a) said antibody or antigen-binding fragment comprises at least one heavy chain complementarity determining region (HCDR1, HCDR2 and/or HCDR3) comprising an amino acid sequence selected from the group consisting of:
HCDR1 SEQ ID NO: 15 or SEQ ID NO: 15 wherein one or two amino acids are conservatively substituted;
HCDR2 SEQ ID NO: 16 or SEQ ID NO: 16 wherein one or two amino acids are conservatively substituted; and
HCDR3 SEQ ID NO: 17 or SEQ ID NO: 17 wherein one or two amino acids are conservatively substituted; and
at least one light chain complementarity determining region (LCDR1, LCDR2 and/or LCDR3) comprising an amino acid sequence selected from the group consisting of:
LCDR1 SEQ ID NO: 20 or SEQ ID NO: 20 wherein one or two amino acids are conservatively substituted;
LCDR2 SEQ ID NO: 21 or SEQ ID NO: 21 wherein one or two amino acids are conservatively substituted; and
LCDR3 SEQ ID NO: 22 or SEQ ID NO: 22 wherein one or two amino acids are conservatively substituted; or
b) said antibody or antigen-binding fragment comprises at least one heavy chain complementarity determining region (HCDR1, HCDR2 and/or HCDR3) comprising an amino acid sequence selected from the following:
HCDR1 SEQ ID NO: 25 or SEQ ID NO: 25 where one or two amino acids are conservatively substituted;
HCDR2 SEQ ID NO: 26 or SEQ ID NO: 26 where one or two amino acids are conservatively substituted; and
HCDR3 SEQ ID NO: 27 or SEQ ID NO: 27 where one or two amino acids are conservatively substituted; and
at least one light chain complementarity determining region (LCDR1, LCDR2 and/or LCDR3) comprising an amino acid sequence selected from the group consisting of:
LCDR1 SEQ ID NO: 30 or SEQ ID NO: 30 wherein one or two amino acids are conservatively substituted;
LCDR2 SEQ ID NO: 31 or SEQ ID NO: 31 wherein one or two amino acids are conservatively substituted; and
LCDR3 SEQ ID NO: 32 or SEQ ID NO: 32 wherein one or two amino acids are conservatively substituted.

13. The antibody or antigen-binding fragment of claim 12 wherein said antibody has the epitopic specificity of monoclonal antibody 5E4 or monoclonal antibody 8D5.

14. An immunoglobulin heavy chain or antigen-binding portion thereof comprising:
a) three heavy chain complementarity determining regions (HCDR1, HCDR2 and HCDR3) comprising the following amino acid sequences:
HCDR1 SEQ ID NO: 15 or SEQ ID NO: 15 wherein one or two amino acids are conservatively substituted;
HCDR2 SEQ ID NO: 16 or SEQ ID NO: 16 wherein one or two amino acids are conservatively substituted; and
HCDR3 SEQ ID NO: 17 or SEQ ID NO: 17 wherein one or two amino acids are conservatively substituted; or
b) three heavy chain complementarity determining regions (HCDR1, HCDR2 and HCDR3) comprising the following amino acid sequences:
HCDR1 SEQ ID NO: 25 or SEQ ID NO: 25 wherein one or two amino acids are conservatively substituted;
HCDR2 SEQ ID NO: 26 or SEQ ID NO: 26 wherein one or two amino acids are conservatively substituted; and
HCDR3 SEQ ID NO: 27 or SEQ ID NO: 27 wherein one or two amino acids are conservatively substituted,
wherein an antibody comprising said heavy chain or antigen-binding portion thereof and a complementary light chain or antigen-binding portion of a complementary light chain binds an αE integrin.

15. An isolated and/or recombinant nucleic acid comprising a nucleotide sequence that encodes the immunoglobulin heavy chain or antigen-binding portion thereof of claim 10 or 14.

16. An expression construct comprising a recombinant nucleic acid comprising a nucleotide sequence that encodes the immunoglobulin heavy chain or antigen-binding portion thereof of claim 10 or 14.

17. A host cell comprising a recombinant nucleic acid encoding the immunoglobulin heavy chain or antigen-binding portion thereof of claim 10 or 14.

18. An immunoglobulin light chain or antigen-binding portion thereof comprising:
a) three light chain complementarity determining regions (LCDR1, LCDR2 and LCDR3) comprising the following amino acid sequences:
LCDR1 SEQ ID NO: 20 or SEQ ID NO: 20 wherein one or two amino acids are conservatively substituted;
LCDR2 SEQ ID NO: 21 or SEQ ID NO: 21 wherein one or two amino acids are conservatively substituted; and
LCDR3 SEQ ID NO: 22 or SEQ ID NO: 22 wherein one or two amino acids are conservatively substituted; or
b) three light chain complementarity determining regions (LCDR1, LCDR2 and LCDR3) comprising the following amino acid sequences:
LCDR1 SEQ ID NO: 30 or SEQ ID NO: 30 wherein one or two amino acids are conservatively substituted;
LCDR2 SEQ ID NO: 31 or SEQ ID NO: 31 wherein one or two amino acids are conservatively substituted; and
LCDR3 SEQ ID NO: 32 or SEQ ID NO: 32 wherein one or two amino acids are conservatively substituted,
wherein an antibody comprising said light chain or antigen-binding portion thereof and a complementary heavy chain or antigen-binding portion of a complementary heavy chain binds an αE integrin.

19. An isolated and/or recombinant nucleic acid comprising a nucleotide sequence that encodes the immunoglobulin light chain or antigen-binding portion thereof of Claim 11 or 18.

20. An expression construct comprising a recombinant nucleic acid comprising a nucleotide sequence that encodes the immunoglobulin light chain or antigen-binding portion thereof of claim 11 or 18.

21. A host cell comprising a recombinant nucleic acid encoding the immunoglobulin light chain or antigen-binding portion thereof of claim 11 or 18

22. An isolated cell that produces the antibody or antigen-binding fragment of claim 1, 8 or 12.

23. An antibody or antigen-binding fragment thereof, wherein said antibody or fragment is selected from the group consisting of:
a) the antibody produced by hybridoma 3G6 (ATCC Accession No. PTA-4201) or an antigen-binding fragment thereof;
b) the antibody produced by hybridoma 5E4 (ATCC Accession No. PTA-4202) or an antigen-binding fragment thereof;
c) the antibody produced by hybridoma 8D5 (ATCC Accession No. PTA-4203) or an antigen-binding fragment thereof;
d) the antibody produced by CHO 3G6 C1.2D6 (ATCC Accession No. PTA-4204) or an antigen-binding fragment thereof; and
e) the antibody produced by CHO 5G4 A1.2C12 (ATCC Accession No. PTA-4205) or an antigen-binding fragment thereof.

24. Hybridoma 3G6 (ATCC Accession No. PTA-4201), Hybridoma 5E4 (ATCC Accession No. PTA-4202), Hybridoma 8D5 (ATCC Accession No. PTA-4203), CHO 3G6 C1.2D6 (ATCC Accession No. PTA-4204) or CHO 5G4 A1.2C12 (ATCC Accession No. PTA-4205).

25. A composition comprising the antibody or antigen-binding fragment of claim 1, 8 or 12 and a physiologically acceptable carrier.

26. A method for treating a subject having an inflammatory disorder, comprising administering to said subject an effective amount of an antibody or antigen-binding fragment thereof which specifically binds an activated αE integrin, wherein said antibody or antigen-binding fragment binds an activation-induced epitope on integrin αE chain (CD103).

27. The method of claim 30 wherein the inflammatory disorder is an inflammatory bowel disease.

28. A method for treating a subject having an inflammatory disorder, comprising administering to said subject an effective amount of an antibody or antigen-binding fragment thereof which binds an αE integrin and inhibits binding of ligand to said αE integrin, wherein:
a) said antibody or antigen-binding fragment comprises at least one heavy chain complementarity determining region (HCDR1, HCDR2 and/or HCDR3) comprising an amino acid sequence selected from the group consisting of:
HCDR1 SEQ ID NO: 5 or SEQ ID NO: 5 wherein one or two amino acids are conservatively substituted;
HCDR2 SEQ ID NO: 6 or SEQ ID NO: 6 wherein one or two amino acids are conservatively substituted; and
HCDR3 SEQ ID NO: 7 or SEQ ID NO: 7 wherein one or two amino acids are conservatively substituted; and
at least one light chain complementarity determining region (LCDR1, LCDR2 and/or LCDR3) comprising an amino acid sequence selected from the group consisting of:
LCDR1 SEQ ID NO: 10 or SEQ ID NO: 10 where one or two amino acids are conservatively substituted;
LCDR2 SEQ ID NO: 11 or SEQ ID NO: 11 where one or two amino acids are conservatively substituted; and
LCDR3 SEQ ID NO: 12 or SEQ ID NO: 12 where one or two amino acids are conservatively substituted; or
b) said antibody or antigen-binding fragment comprises at least one heavy chain complementarity determining region (HCDR1, HCDR2 and/or HCDR3) comprising an amino acid sequence selected from the group consisting of:
HCDR1 SEQ ID NO: 15 or SEQ ID NO: 15 wherein one or two amino acids are conservatively substituted;
HCDR2 SEQ ID NO: 16 or SEQ ID NO: 16 wherein one or two amino acids are conservatively substituted; and
HCDR3 SEQ ID NO: 17 or SEQ ID NO: 17 wherein one or two amino acids are conservatively substituted; and
at least one light chain complementarity determining region (LCDR1, LCDR2 and/or LCDR3) comprising an amino acid sequence selected from the following:
LCDR1 SEQ ID NO: 20 or SEQ ID NO: 20 wherein one or two amino acids are conservatively substituted;
LCDR2 SEQ ID NO: 21 or SEQ ID NO: 21 wherein one or two amino acids are conservatively substituted; and
LCDR3 SEQ ID NO: 22 or SEQ ID NO: 22 wherein one or two amino acids are conservatively substituted; or
c) said antibody or antigen-binding fragment comprises at least one heavy chain complementarity determining region (HCDR1, HCDR2 and/or HCDR3) comprising an amino acid sequence selected from the group consisting of:
HCDR1 SEQ ID NO: 25 or SEQ ID NO: 25 wherein one or two amino acids are conservatively substituted;
HCDR2 SEQ ID NO: 26 or SEQ ID NO: 26 wherein one or two amino acids are conservatively substituted; and
HCDR3 SEQ ID NO: 27 or SEQ ID NO: 27 wherein one or two amino acids are conservatively substituted; and
at least one light chain complementarity determining region (LCDR1, LCDR2 and/or LCDR3) comprising an amino acid sequence selected from the group consisting of:
LCDR1 SEQ ID NO: 30 or SEQ ID NO: 30 wherein one or two amino acids are conservatively substituted;
LCDR2 SEQ ID NO: 31 or SEQ ID NO: 31 wherein one or two amino acids are conservatively substituted; and
LCDR3 SEQ ID NO: 32 or SEQ ID NO: 32 wherein one or two amino acids are conservatively substituted.

29. A method for detecting an activated αE integrin comprising contacting a composition comprising an αE integrin with an antibody or antigen-binding fragment thereof which binds an activation-induced epitope on integrin αE chain (CD103) and detecting formation of a complex between said antibody or antigen-binding fragment and said activated αE integrin.
